(19) United States
(12) Patent Application Publication
(10) Pub. No.: US 2023/0047712 A1
(43) Pub. Date: Feb. 16, 2023

(54) Methods of Treatments Based Upon Molecular Response to Treatment

(71) Applicant: The Board of Trustees of the Leland Stanford Junior University, Stanford, CA (US)

(72) Inventors: Christina Curtis, Stanford, CA (US); Katherine McNamara, Stanford, CA (US)

(73) Assignee: The Board of Trustees of the Leland Stanford Junior University, Stanford, CA (US)

(21) Appl. No.: 17/755,519

(22) PCT Filed: Oct. 29, 2020
(86) PCT No.: PCT/US20/58050

§ 371 Date: Apr. 29, 2022

Related U.S. Application Data
(60) Provisional application No. 62/927,557, filed on Oct. 29, 2019.

(51) Int. Cl. C12Q 1/6886; G16B 25/10; G16B 40/00

(52) U.S. Cl. CPC C12Q 1/6886; G16B 25/10; G16B 40/00; C12Q 2600/106; C12Q 2600/158

(57) ABSTRACT

Methods of treatment based on a breast cancer's biomolecule response to targeted treatment are provided. Expression levels of various biomolecules or histological assessment of infiltrating immune cells after initiation of human epidermal growth factor receptor 2 (HER2) targeted treatment can be used to determine whether a breast cancer will achieve a pathologic complete response. Based on likelihood of a pathologic complete response, a breast cancer can be treated accordingly.

### CROSS REFERENCE TO RELATED APPLICATIONS

This application claims priority to U.S. Provisional Application No. 62/927,557, entitled “Methods of Treatments Based Upon Molecular Response to Neoadjuvant Treatment” to Curtis et al., filed Oct. 29, 2019, which is incorporated herein by reference in its entirety.

### STATEMENT REGARDING FEDERALLY SPONSORED RESEARCH OR DEVELOPMENT

This invention was made with Government support under contract CA182514 awarded by the National Institutes of Health. The Government has certain rights in the invention.

### TECHNICAL FIELD

The disclosure is generally directed to methods involving diagnostics and treatments based upon molecular characterization of an individual's breast cancer and molecular response to treatment.

### BACKGROUND

Human epidermal growth factor receptor 2-positive (HER2+) breast cancer is a breast cancer that tests positive for a protein called human epidermal growth factor receptor 2 (HER2), which promotes the growth of cancer cells. HER2+ breast cancer accounts for 15-30% of invasive breast cancers and is associated with an aggressive phenotype. A number of targeted therapies can be used for HER2+ breast cancer, including trastuzumab (Herceptin), lapatinib (Tykerb), neratinib (Nerlynx), pertuzumab (perjeta), and ado-trastuzumab emtansine (T-DM1 or Kadcyla). Targeted therapies are often utilized as neoadjuvant treatments, which are treatments to reduce tumor size prior to surgery.

### SUMMARY

Various embodiments are directed to diagnostics and treatments of breast cancer based on molecular response to targeted treatment. In various embodiments, the cancer's molecular response to a targeted treatment is determined by measuring expression of particular tumor-related or immune-related biomolecules. In various embodiments, a linear model utilized biomolecule expression to determine the likelihood of achieving complete pathologic response to a targeted treatment. In various embodiments, particular treatment regimens are performed based on the likelihood of achieving complete pathologic response.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a flow diagram of a method to treat a breast cancer based upon a classification indicative of pathologic complete response (pCR) in accordance with an embodiment of the invention.

FIG. 2 provides a schematic overview of the discovery and validation cohorts analyzed with the GeoMx™ Digital Spatial Profiling (DSP) technology, utilized in accordance with various embodiments. Patients with invasive HER2+ breast cancer enrolled on the TRIO-US B07 clinical trial were treated with one cycle of the assigned HER2-targeted therapy followed by six cycles of the assigned HER2-targeted treatment plus chemotherapy (docetaxel+carboplatin). Tissue was obtained at three timepoints (pre-treatment, on-treatment, and post-treatment/surgery).

FIG. 3 provides a summary of the clinical characteristics of the TRIO-US B07 DSP discovery cohort, including treatment arm, pathologic complete response (pCR), estrogen receptor (ER) status, and PAM50 status inferred based on pre-treatment bulk expression data, utilized in accordance with various embodiments. Two-way contingency tables compare the distribution of ER status, pCR status, and treatment arm.

FIG. 4 provides a chart indicating pathology-estimated cellularity pre-treatment and on-treatment for the discovery cohort, utilized in accordance with various embodiments. Samples with green shading indicate those used for subsequent analysis. For the pathologic complete response (pCR) column, 0=non-pCR, 1=pCR. For the estrogen receptor (ER) status column, 0=ER-negative, 1=ER-positive. FIG. 4 also provides an example in situ region from case 30 sampled on-treatment, utilized in accordance with various embodiments. While cellularity was estimated to be 0 based on pathology review of a distinct tissue section, tumor regions were identified upon imaging the tissue section used in this analysis.

FIG. 5 provides a schematic summarizing the NanoString Digital Spatial Profiler workflow, utilized in accordance with various embodiments. The slide is stained with the mix of protein antibodies. The antibodies have an indexing oligo attached, which is used for subsequent readout. ROIs (regions of interest) are selected and illuminated using UV (ultraviolet) light. The UV light causes the indexing oligos within the ROI to be cleaved off for collection and per-probe quantification.

FIG. 6 provides a schematic and images depicting regions of interest analyzed, utilized in accordance with various embodiments. Multiple regions of interest (ROIs) per tissue sample were selected based on pancytokeratin enrichment (panCK-E) and subject to spatial proteomic profiling of 40 tumor and immune markers. Protein counts were measured within phenotypic regions corresponding to the PanCK-E masks that includes tumor cells and co-localized immune cells and separately for the inverted mask corresponding to panCK-negative regions.

FIG. 7 provides sample images depicting multiple regions of interest, utilized in accordance with various embodiments. The location of spatially separated ROIs within tissue specimens for a representative pCR case (69) and a demonstrative non-pCR case (58). An average of 4 ROIs were profiled per tissue (range: 1-7).

FIG. 8 provides a correlation plot comparing Ki67 percent positive (evaluated using IHC) with normalized DSP Ki67 expression (averaged across all ROIs within a distinct tissue slice from the same case and timepoint), generated in accordance with various embodiments. A total of 42 biopsies (24 pre-treatment and 18 on-treatment) with paired Ki67 IHC and DSP data were utilized in this analysis. Pearson correlation coefficient and corresponding p-value are also noted. FIG. 8 also provides a boxplot comparing normalized DSP Her2 expression (averaged across all ROIs from the same case and timepoint) between cases that exhibited strong (3+) IHC Her2 staining (using a distinct tissue slice from the same case and timepoint) or weaker (0-2) IHC Her2 stating, generated in accordance with various embodiments. A total of 44 biopsies (23 pre-treatment and 21 on-treatment) with paired Her2 IHC and DSP data were utilized in this analysis. A Wilcoxon test was used to assess significance.

FIG. 9 provides a pairwise correlation of pre-treatment protein marker expression across all ROIs in the discovery cohort, utilized in accordance with various embodiments. Black squares indicate probes in the same hierarchical cluster.

FIG. 10 provides a chart depicting inter-tumor and intra-tumor variability in HER2 and CD45 protein expression in untreated HER2-positive breast tumors from the discovery cohort, where each point corresponds to an ROI, utilized in accordance with various embodiments. Clinical characteristics, including pCR status, estrogen receptor (ER) status, and PAM50 subtype (based on gene expression profiling) are indicated.

FIGS. 11A and 11B provide violin plots depicting CD45 values and CD56 values from the Digital Spatial Profiling (DSP) protein data on-treatment (FIG. 11A) and pretreatment (FIG. 11B) in the pCR cases versus the non-pCR cases, utilized in accordance with various embodiments. Each point represents the average probe values for all panCK-enriched ROIs for that case On-treatment. The p-value was derived using a linear mixed-effect model over the multi-region data with blocking by patient. For each violin plot, the white box represents the interquartile range and the black lines extending from the white box represent 1.5× the interquartile range. Analyses based on the discovery cohort.

FIG. 12 provides a volcano plot demonstrating treatment-associated changes based on comparison of pre-treatment versus on-treatment protein marker expression levels in pancytokeratin-enriched (PanCK-E) regions, utilized in accordance with various embodiments. Significance, −log10(FDR adjusted p-value), is indicated along the y-axis.

FIG. 13 provides a volcano plot demonstrating treatment-associated changes based on comparison of pre-treatment versus on-treatment bulk RNA expression levels, utilized in accordance with various embodiments. RNA transcripts with corresponding Digital Spatial Profiling (DSP) protein markers were used in this analysis. Significance, −log10 (FDR adjusted p-value), is indicated along the y-axis. Analyses based on the discovery cohort.

FIG. 14 provides a table of pairing of protein antibodies and gene names used in comparative analyses between DSP and bulk expression data, utilized in accordance with various embodiments.

FIG. 15 provides a volcano plot demonstrating treatment-associated changes based on comparison of pre-treatment versus on-treatment protein marker expression levels in pancytokeratin-enriched (PanCK-E) regions in the trastuzumab-treated cases (arms 1 and 3, n=23). Significance, −log10(FDR adjusted p-value), is indicated along the y-axis, utilized in accordance with various embodiments. Analyses based on the discovery cohort.

FIGS. 16A and 16B provide volcano plots demonstrating treatment-associated changes in pCR versus non-pCR cases, utilized in accordance with various embodiments.

FIGS. 17A and 17B provide pairwise correlations of protein markers in pCR versus non-pCR cases, utilized in accordance with various embodiments. Black squares demarcate hierarchical clusters.

FIG. 18 provides waterfall plots illustrating treatment-associated changes (pre-treatment to on-treatment) in ER+ and ER− cases based on protein expression, utilized in accordance with various embodiments.

FIG. 19 provides waterfall plots illustrating treatment-associated changes (pre-treatment to on-treatment) based on in pancytokeratin-enriched (PanCK-E) regions from DSP protein expression data, utilized in accordance with various embodiments. Input data was stratified both by estrogen receptor (ER) status and pathologic complete response (pCR) outcome. Analyses based on the discovery cohort.

FIG. 20 provides waterfall plots illustrating treatment-associated changes in DSP protein expression (pre-treatment to on-treatment) in HER2-enriched and non-HER2-enriched cases (n=7 normal-like, n=2 luminal B, n=2 basal, n=1 luminal A), utilized in accordance with various embodiments. Analyses were performed in the discovery cohort.

FIG. 21 waterfall plots illustrating treatment-associated changes (pre-treatment to on-treatment) based on in pancytokeratin-enriched (PanCK-E) regions from the DSP protein expression data, utilized in accordance with various embodiments. Samples were stratified both by PAM50 status (Her2-Enriched or other) and pathologic complete response (pCR) outcome.

FIG. 22 provides waterfall plots, generated using pancytokeratin-enriched (PanCK-E) regions from DSP protein expression data, illustrating treatment-associated changes (pre-treatment to on-treatment) when only one region is used to profile each sample (averaged across 100 iterations of random samples of a single region per timepoint), rather than the 2-7 regions from each sample used in other analyses, utilized in accordance with various embodiments. The upper plot is for all patients, and the lower plots are stratified by pathologic complete response (pCR) status. Analyses based on the discovery cohort.

FIG. 23 provides a volcano plot demonstrating treatment-associated changes from pre-treatment to surgery in tumors that did not undergo pathologic complete response (pCR) using DSP protein expression levels in pancytokeratin-enriched (PanCK-E) regions, utilized in accordance with various embodiments. Significance, −log10(FDR adjusted p-value), is indicated along the y-axis. Analyses based on the discovery cohort.

FIG. 24 provides representative in situ images of ROIs from two cases and quantification of HER2 and CD45 protein levels (log2 normalized) in panCK-enriched regions, utilized in accordance with various embodiments.

FIG. 25 provides a chart showing comparison of DSP HER2 protein levels pre-treatment and on-treatment for all regions profiled per case per timepoint, utilized in accordance with various embodiments.

FIG. 26 provides a table showing comparison of the mean squared error in DSP HER2 protein expression pre-treatment versus on-treatment within and between patients, utilized in accordance with various embodiments. P-values are based on a two-sided paired Wilcoxon signed rank test. Analyses are based on the discovery cohort.

FIG. 27 provides charts depicting pretreatment versus on-treatment heterogeneity for each DSP tumor and immune marker, utilized in accordance with various embodiments. P-values are based on a two-sided paired Wilcoxon signed rank test. Analyses are based on the discovery cohort.

FIG. 28 provides charts depicting pre, on-, and post-treatment heterogeneity for each DSP protein marker in non-pCR cases (patients with tumor cells present at surgery), utilized in accordance of various embodiments. Analyses based on the discovery cohort.

FIG. 29 provides charts depicting on-treatment heterogeneity in DSP protein markers for pCR and non-pCR cases, utilized in accordance with various embodiments.

FIG. 30 provides charts depicting pretreatment treatment heterogeneity in DSP protein marker expression in pCR and non-pCR cases, utilized in accordance with various embodiments. Heterogeneity was calculated as the mean squared error within patients based on analysis of variance. P-values are based on a two-sided Wilcoxon matched-pair signed rank test. Analyses based on the discovery cohort.

FIG. 31 provides a schematic of digital spatial profiling (DSP), which was performed on multiple regions of interest (ROIs) per tissue sample, utilized in accordance with various embodiments. Protein counts were measured within phenotypic regions corresponding to the panCK-enriched (tumor-enriched) masks that include tumor cells and co-localized immune cells and separately for the inverted mask corresponding to panCK-negative (tumor microenvironment, TME) regions.

FIGS. 32A, 32B, and 32C provide waterfall plots of DSP protein data reveal differences in immune marker expression between immune-dense panCK-enriched regions and the surrounding panCK-negative regions profiled pre-treatment, on-treatment, and post-treatment, utilized in accordance with various embodiments. Heterogeneity was calculated as the mean squared error within patients based on analysis of variance. P-values are based on a two-sided paired Wilcoxon signed rank test. Analyses are based on the discovery cohort.

FIGS. 33A and 33B provide waterfall plots, generated using the DSP protein data, comparing immune marker expression between the panCK-enriched regions and the surrounding panCK-negative regions pre-treatment and on-treatment, in pCR (n=14) and non-pCR cases (n=14), utilized in accordance with various embodiments. Pre-treatment, the correlation between immune marker fold-change values in the pCR and non-pCR cases was 0.98 indicating similar immune distribution across the panCK-enriched regions and surrounding microenvironment regardless of pCR outcome and this correlation remained high on-treatment (0.95). Analyses based on the discovery cohort.

FIGS. 34A and 34B provide waterfall plots, generated using DSP protein data, comparing immune marker expression between the panCK-enriched regions and the surrounding panCK-negative regions pre-treatment and on-treatment, in ER-positive cases (n=14), and ER-negative cases (n=14), utilized in accordance with various embodiments. Analyses based on the discovery cohort.

FIG. 35 provides multiplex immunohistochemistry (mIHC) images showing the distribution of HER2, CD45, and CD8 signal in representative tissue stamps pre-treatment and on-treatment, utilized in accordance with various embodiments. The panCK mIHC channel (not shown) was used to generate the panCK mask and the tissue mask (outlined in yellow). IHC marker expression levels for HER2, CD45, and CD8 were quantified for the whole tissue section (across all digitized sub-images) and within the panCK-enriched tumor regions (across all digitized sub-images).

FIG. 36 provides an illustration of panCK-enriched binary masks and perimetric complexity-based quantification of the tumor-microenvironment border, utilized in accordance with various embodiments.

FIG. 37 provides a violin plot depicting comparison of perimetric complexity values pre-treatment between pCR cases and non-pCR cases, utilized in accordance with various embodiments. P-values computed with a linear model, blocked by patient. Analyses are based on the discovery cohort.

FIG. 38 provides a violin plot depicting comparison of pre-treatment versus on-treatment perimetric complexity values, utilized in accordance with various embodiments. PanCK-enriched ROIs were used to quantify perimetric complexity. P-values computed with a linear model, blocked by patient. Analyses are based on the discovery cohort.

FIG. 39 provides a plot depicting Spearman correlation between the DSP protein expression values and perimetric complexity per region of interest (ROI) in the pre-treatment and on-treatment tissue specimens from the discovery cohort, utilized in accordance with various embodiments. Significantly correlated probes: p-value<0.05 are denoted by an asterisk. Correlation plot for Ki-67, the marker with the highest correlation with perimetric complexity, where each dot represents an individual ROI.

FIG. 40 provides area under the receiver operating characteristics (AUROC) performance of various models were compared using nested cross-validation with Holm-Bonferroni correction for multiple hypotheses in the discovery (training) cohort, generated in accordance with various embodiments. Receiver operating characteristic (ROC) curves were generated using cases with DSP panCK-enriched data from both the pre-treatment and on-treatment timepoints (n=23). ROC curves and statistical comparison of L2-regularized classifiers trained using DSP protein marker mean values (averaged across ROIs) pre-treatment, on-treatment and the combination of pre-treatment and on-treatment (“On- +Pre-treatment”).

FIG. 41 provides area under the receiver operating characteristics (AUROC) performance of various models were compared using nested cross-validation with Holm-Bonferroni correction for multiple hypotheses in the discovery (training) cohort, generated in accordance with various embodiments. Receiver operating characteristic (ROC) curves were generated using cases with DSP panCK-enriched data from both the pre-treatment and on-treatment timepoints (n=23). ROC curves and statistical comparison of DSP protein On- plus Pre-treatment L2-regularized classifiers trained using all marker, tumor marker, and immune marker mean values. Cross-region mean marker values from both the pre-treatment and on-treatment timepoints were used in this analysis.

FIG. 42 provides area under the receiver operating characteristics (AUROC) performance (using nested cross-validation with Holm-Bonferroni correction for multiple hypotheses) comparing DSP protein on- plus pre-treatment L2-regularized classifiers trained using marker means versus marker standard error of the mean (SEM) for tumor markers and immune markers, generated in accordance with various embodiments. Model comparisons were performed in the discovery cohort.

FIG. 43 provides area under the receiver operating characteristics (AUROC) performance of various models were compared using nested cross-validation with Holm-Bonferroni correction for multiple hypotheses in the discovery (training) cohort, generated in accordance with various embodiments. Receiver operating characteristic (ROC) curves were generated using cases with DSP panCK-enriched data from both the pre-treatment and on-treatment timepoints (n=23). ROC curves and statistical comparison of the On-plus Pre-treatment DSP protein L2-regularized classifier to a model trained using ER and PAM50 status. These two models were compared to a model that incorporates On- plus Pre-treatment DSP protein data, ER and PAM50 status.

FIG. 44 provides receiver operating characteristic (ROC) curves and AUROC (Area Under Receiver Operating Characteristic) quantification for the On- plus Pre-treatment DSP protein L2-regularized classifier using all 40 markers compared to other models, generated in accordance with various embodiments. ROC and statistical comparison to a model trained using ER, PAM50 status, and strong (3+) HER2 IHC (immunohistochemistry) staining status, pre-treatment, in n=19 patients with all data available. These two models are also compared to a model that incorporates On- plus Pre-treatment DSP protein data, ER, PAM50 status, and HER2 IHC staining status. ROC and statistical comparison to a model trained using ER, PAM50 status, and HER2 FISH (fluorescence in situ hybridization) ratio, pre-treatment, in n=21 patients with all data available. These two models are also compared to a model that incorporates On- plus Pre-treatment DSP protein data, ER, PAM50 status, and HER2 FISH ratio. ROC and statistical comparison to a model trained using on-treatment stromal tumor infiltrating lymphocytes (TILs) in n=16 patients with all data available. These two models are also compared to a model that incorporates On- plus Pre-treatment DSP protein data and on-treatment TILs.

FIG. 45 provides ROC and statistical comparison of On- plus Pre-treatment L2-regularized classifiers trained using DSP protein marker mean values versus bulk RNA expression using RNA transcripts corresponding to the DSP protein markers, generated in accordance with various embodiments. ROC curves were generated using cases with DSP panCK-enriched data and bulk expression data from both the pre-treatment and on-treatment timepoints (n=21).

FIG. 46 provides a plot depicting Spearman correlation between DSP protein probes (averaged across all ROIs per case) and bulk RNA transcripts corresponding to these markers pre-treatment, utilized in accordance embodiments. Significantly correlated probes (with p-value<0.05) are indicated by an asterisk. Two exemplary correlation plots are shown, where each dot represents a single case. Analyses based on the discovery cohort.

FIG. 47 provides a table summarizing the clinical characteristics for the TRIO-US B07 clinical trial Digital Spatial Profiling (DSP) validation cohort used for model testing, utilized in accordance with various embodiments. Treatment arm, pathologic complete response (pCR), estrogen receptor (ER) status, and PAM50 status inferred based on pre-treatment bulk expression data are included. Two-way contingency tables compare the distribution of ER status, pCR status, and treatment arm.

FIG. 48 provides a volcano plot demonstrating treatment-associated changes based on comparison of pre-treatment versus on-treatment protein marker expression levels in pancytokeratin-enriched (PanCK-E) regions in the validation cohort, utilized in accordance with various embodiments. Significance, −log10(FDR adjusted p-value), is indicated along the y-axis.

FIG. 49 provides volcano plots demonstrating treatment-associated changes in pCR versus non-pCR cases in the PanCK-E regions in the validation cohort, utilized in accordance with various embodiments. Significance, −log10(FDR adjusted p-value), is indicated along the y-axis.

FIG. 50 provides receiver operating characteristic (ROC) curves for On- plus Pre-treatment DSP protein L2-regularized classifier in the discovery (training) cohort (n=23, assessed via cross-validation) and the validation (test) cohort (n=28, assessed via train-test) using the 40-plex DSP protein marker panel, generated in accordance with various embodiments.

FIG. 51 provides a plot depicting coefficients for each of the 40 markers in the L2-regularized On- plus Pre-treatment DSP protein model, trained in the discovery cohort, and tested in the validation cohort, generated in accordance with various embodiments.

FIG. 52 provides receiver operating characteristic (ROC) curves for On- plus Pre-treatment DSP protein L2-regularized classifier in the discovery cohort using all cases with panCK-enriched data from both timepoints (n=23) and in the subset of cases treated with trastuzumab or trastuzumab+lapatinib (n=19), generated in accordance with various embodiments. Model performance was assessed via cross-validation using the 40 DSP protein markers profiled in both cohort

FIG. 53 provides a correlation plot comparing the marker coefficients for the On-plus Pre-treatment DSP protein trained using all cases in the discovery cohort and using only those cases treated with trastuzumab (arms 1 and 3), generated in accordance with various embodiments.

FIG. 54 provides a plot depicting coefficients for each marker in the L2-regularized On- plus Pre-treatment DSP protein model, trained using only those cases treated with trastuzumab (arms 1 and 3), generated in accordance with various embodiments.

FIG. 55 provides ROC curves for On- plus Pre-treatment L2-regularized classifier in the discovery (training) cohort (n=23, assessed via cross-validation) and the validation (test) cohort (n=28, assessed via train-test) using HER2 and CD45 from the DSP protein marker panel, generated in accordance with various embodiments.

FIG. 56 provides a chart depicting coefficients for each marker in the L2-regularized On- plus Pre-treatment DSP protein model, trained using only CD45 and Her2, generated in accordance with various embodiments.

FIG. 57 provides ROC curves for On- plus Pre-treatment L2-regularized classifier in the discovery (training) cohort (n=23, assessed via cross-validation) and the validation (test) cohort (n=28, assessed via train-test) using CD45 from the DSP protein marker panel, generated in accordance with various embodiments.

FIG. 58 provides a chart depicting coefficients for each marker in the L2-regularized On- plus Pre-treatment DSP protein model, trained using only CD45, generated in accordance with various embodiments.

FIG. 59 provides ROC curves for On-treatment L1-regularized classifier in the discovery (training) cohort (n=23, assessed via cross-validation) and the validation (test) cohort (n=28, assessed via train-test) using CD45 from the DSP protein marker panel, generated in accordance with various embodiments.

FIG. 60 provides a table of markers with a signal to noise ratio (SNR)<3 in the discovery cohort indicated by a caret (\hat{ }) and those with an SNR<3 in the validation cohort are indicated with an asterisk (*), utilized in accordance with various embodiments.

### DETAILED DESCRIPTION

Turning now to the drawings and data, methods of predicting pathologic complete response (pCR) and treating HER2+ breast cancer based upon the cancer's predicted pCR are provided. As understood in the field, a pathologic complete response is defined as a disappearance of all invasive cancer in the breast tissue after completion of neoadjuvant chemotherapy. Numerous embodiments are directed towards evaluating one or more tumor biopsies of a patient that has been diagnosed with breast cancer. In some embodiments, the individual is diagnosed with HER2+ breast cancer. In some embodiments, molecular evaluation of a tumor biopsy occurs prior to any treatment (i.e., pretreatment). In some embodiments, molecular evaluation of a tumor biopsy occurs after initiation of targeted therapy (also referred herein to as the on-treatment time-point), which can occur during a neoadjuvant treatment. In some embodiments, molecular evaluation of a tumor biopsy occurs after soon after initiation of targeted therapy (e.g., about: 48 hours, 72 hours, 96 hours, 120 hours, 144 hours, or 168 hours after initiation). In some embodiments, molecular evaluation of a tumor biopsy occurs after soon after completion of the first cycle of targeted therapy (e.g., about: 48 hours, 72 hours, 96 hours, 120 hours, 144 hours, or 168 hours after completion of the first cycle). In some embodiments, molecular evaluation of a tumor biopsy occurs both prior to any treatment and after initiation of targeted therapy. In some embodiments, biomolecule expression after initiation of targeted therapy is used to predict pCR. In some of these embodiments, the change of biomolecule expression that occur prior to any treatment and after one cycle of targeted therapy is used to predict pCR. In some embodiments, histological assessment of immune infiltrating cells after initiation of targeted therapy is used to predict pCR.

In accordance with multiple embodiments, treatment is determined by the likelihood of response to neoadjuvant therapy to achieve pCR, which can be utilized to escalate or deescalate treatment. In several embodiments, neoadjuvant therapy is a used to reduce tumor size prior to a subsequent therapy (e.g., surgery). In some embodiments when neoadjuvant therapy is predicted to achieve pCR, a deescalated treatment is utilized, such as (for example) targeted treatment directed at HER2 is administered without generalized chemotherapy (i.e., non-targeted chemotherapy). Targeted treatments include (but not limited to) trastuzumab, lapatinib, pertuzumab, T-DM1, and any combination thereof. In some embodiments, a targeted chemotherapeutic agent is used (e.g., ado-trastuzumab emtansine (T-DM1)). In many embodiments, when neoadjuvant therapy is not predicted to result in a pCR, an escalated treatment regimen can be administered, such as (for example) targeted treatment with chemotherapy and/or dual targeted-therapies, including in the neoadjuvant and/or adjuvant settings. Chemotherapeutics include (but not limited to) taxanes including paclitaxel (Taxol), anthracyclines including doxorubicin (Adriamycin), cyclophosphamide, and any combination thereof.

Based on recent discoveries, the link between expression of particular tumor and immune biomolecules after initiation of targeted therapy and pCR is now appreciated, indicating courses of treatment and surveillance. Accordingly, embodiments are directed to classifying breast cancer based on its likelihood to achieve pCR via a targeted treatment in order to determine a treatment regimen that is well-suited for that breast cancer.

### Treatment of Breast Cancer Determined by Molecular Response

A number of embodiments are directed to classifying a breast cancer on its likelihood of pCR after target treatment (especially neoadjuvant targeted treatment). In several embodiments, a breast cancer classification is based on biomolecule expression in a tumor biopsy as determined after initiation of targeted treatment. Particular biomolecule expression patterns, in accordance with several embodiments, indicate whether a breast cancer has a high likelihood to achieve pCR. In some embodiments, a breast cancer classification is based on histological assessment of immune infiltrating cells after initiation of targeted therapy. In some embodiments, biomolecule expression and/or assessment of immune infiltrating cells is determined pretreatment and after initiation of targeted treatment such that change of expression and/or change of immune cell infiltration can be determined. Based on a classification of pCR likelihood, a number of embodiments determine a course of treatment for a breast cancer.

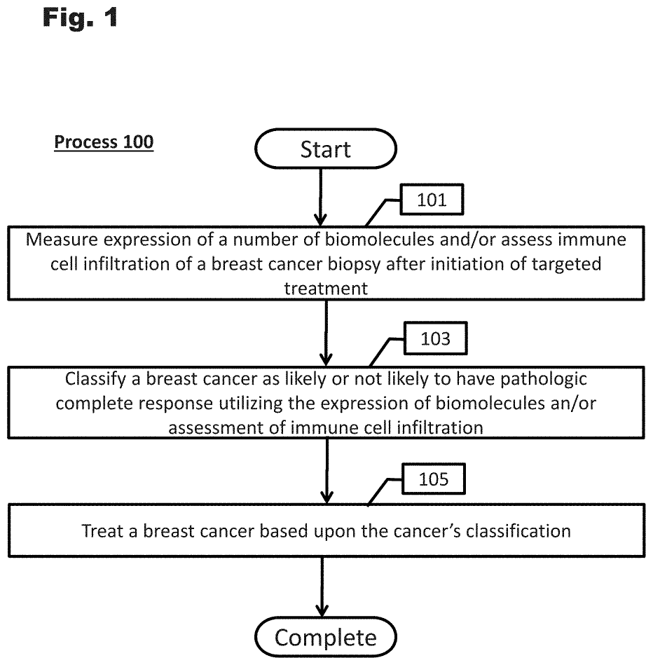

Provided in FIG. 1 is a method to classify an individual's breast cancer based on expression of biomolecules and/or infiltration of immune cells after initiation of targeted therapy, which is indicative of likelihood of pCR and thus the cancer is treated accordingly. In some embodiments, the breast cancer is HER2+. Process 100 begins with measuring 101 expression of a number of biomolecules and/or assessing immune cell infiltration of a breast cancer after initiation of targeted treatment. In several embodiments, a breast cancer biopsy is utilized to perform biomolecule expression and/or immune cell infiltration analysis. In some embodiments, biomolecule expression and/or immune cell infiltration analysis is performed on particular regions of interest of the biopsy. In some embodiments, biomolecule expression and/or immune cell infiltration analysis is performed on regions where tumor cells and infiltrated immune cells are interacting. In some embodiments, biomolecule expression and/or immune cell infiltration analysis is performed on regions having pancytokeratin-positive (panCK+) tumor cells, which is indicative of infiltrated immune cells that are directly interacting with the tumor cells. In some embodiments, biomolecule expression and/or immune cell infiltration analysis is performed on regions having CD45-positive (CD45+) immune cells, which is a pan-leukocyte marker.

In a number of embodiments, biomolecule expression and/or immune cell infiltration is determined after of the initiation of targeted treatment. It is advantageous to determine biomolecule expression and/or immune cell infiltration during early treatment such that an appropriate treatment course can be determined and administered. In various embodiments, biomolecule expression and/or immune cell infiltration is determined after initiation of treatment and prior to completion of one cycle, after one cycle of treatment and prior to a second cycle of treatment, after at least one cycle of treatment and prior to a third cycle of treatment, after at least one cycle of treatment and prior to a fourth cycle of treatment, or any combination thereof. In some embodiments, biomolecule expression and/or immune cell infiltration is determined pretreatment, prior to any targeted treatments. When biomolecule expression and/or immune cell infiltration is determined at multiple time points, in accordance with multiple embodiments, the dynamics of biomolecule expression can be determined. For instance, in some embodiments, the change in biomolecule expression and/or the change in immune cell infiltration from pretreatment to after the first cycle of treatment. In some embodiments, a linear mixed-effects model is utilized to quantify the dynamics of biomolecule expression from pretreatment to after the first cycle of treatment. As stated previously, targeted treatments include (but not limited to) trastuzumab, lapatinib, pertuzumab, T-DM1, and any combination thereof.

It is now understood, and as described herein, that a number of biomolecules provide an indication of whether a breast cancer is likely to achieve pCR. In general, biomolecules associated with HER2 signaling and immune activation can be detected and measured. Based on recent findings, measurements of the following HER2 signaling pathway biomolecules (RNA or protein) were found to provide an indication of whether a breast cancer will achieve a pCR (after a full course of neoadjuvant therapy): HER2, AKT/p-AKT, S6/p-S6, PTEN, p-ERK, and p-STAT3. Likewise, measurements of biomolecules expressed within epithelial tumor tissue were found to provide an indication of whether a breast cancer will achieve a pCR (after a full course of neoadjuvant therapy): PanCK, Ki67, and Beta-catenin. Generally, decreases in HER2 signaling pathway biomolecules are indicative of pCR. Likewise, measurements of the following immune response and activation biomolecules (RNA or protein) were found to provide an indication of whether a breast cancer will achieve a pCR (after a full course of neoadjuvant therapy): CD45, CD3, CD4, CD8, CD27, CD44, CD45RO, OX40L, ICOS, Granzyme B, CD19, CD11c, CD163, CD68, CD56, CD66B, CD14, STING, PD1/PDL1, B7-H3, B7-H4, IDO-1, Lag3, and VISTA. Generally, increases of immune response and activation biomolecules are indicative of pCR. In addition, measurements of the following cell survival biomolecules (RNA or protein) were found to provide an indication of whether a breast cancer will achieve a pCR (after a full course of neoadjuvant therapy): Beta-2 microglobulin and Bcl-2. It should be understood that other biomolecule measurements can be performed that provide an indication of whether a breast cancer will achieve pCR.

It has been determined that a number of biomolecules provide great contribution to prediction of pCR status, including HER2, Ki67, pS6, CD45, CD56, STING, VISTA, and CD66B. Accordingly, in several embodiments, at least one or more of biomolecule expression measurements of HER2, Ki67, pS6, CD45, CD56, STING, VISTA, and CD66B is determined to predict pCR status.

It is now further understood that infiltration of immune cells into tumoral tissue also provides an indication of whether a breast cancer is likely to achieve pCR. In general, lymphocytes and other immune cells can be assessed by histology or immunostaining techniques. In some embodiments, cancer biopsies can be stained with hematoxylin and eosin (H&E) and infiltrating immune cells can be counted. In some embodiments, H&E stained cancer biopsies are assessed to quantify infiltration of stromal tumor infiltrating lymphocytes (sTILs) or intratumoral lymphocytes (iTu-Ly). In some embodiments, cancer biopsies can be assessed by immunostaining with an anti-CD45 antibody and/or an anti-CD56 to determine the number of infiltrating lymphocytes. Immunostaining can be performed in a number ways, including (but not limited to) chromogenic immunohistochemistry (IHC), immunofluorescence, or elemental isotope staining (e.g., antibodies labeled elemental isotopes).

In many embodiments, biomolecule expression measurements and/or assessment of immune cell infiltration are performed on at least one region of a tumor biopsy. In some embodiments, biomolecule expression measurements and/or assessment of immune cell infiltration are performed on at least two regions of a tumor biopsy and the measurements are combined in an appropriate method (e.g., sum, average, median, standard error, standard deviation, weighted). Regions of interest within a tumor biopsy to perform biomolecule expression measurements can be determined by any appropriate method. In some embodiments, regions of interest are determined by identification of tumor cells, identification of infiltrating immune cells, or a combination thereof. In some embodiments, regions of interest are determined by panCK+ expression. In some embodiments, regions of interest are determined by CD45+ expression.

As depicted, process 100 also classifies 103 a breast cancer as likely or not likely to have a pCR after targeted treatment utilizing the biomolecule expression measurements and/or infiltrating immune cell data as input into a classifier model. Any appropriate classifier can be utilized that can provide a classification of pCR utilizing biomolecule expression measurements and/or infiltrating immune cell data. In some embodiments, the classifier is a regression model. Regression models include (but not limited to) linear, logistic, polynomial, ridge, stepwise, LASSO, elastic net, L1 regularized, L2 regularized, and any combination thereof. In various embodiments, a classifier is one of: generalized linear model (GLM), ordinary least squares, random forests, decision trees or neural networks. Models can be trained utilizing collections of individuals that have had their biomolecules measured and/or infiltrating immune cell data assessed at one or more time points and their pCR determined after a course of treatment (especially neoadjuvant treatment). Accordingly, in various embodiments, collections of individuals with breast cancer (e.g., HER2+) that have had their biomolecules measured and/or infiltrating immune cell data assessed from a tumor biopsy at baseline and/or after initiation of targeted treatment can be utilized to train a model to predict pCR. In some embodiments, a classifier model is trained to determine whether an individual should receive a deescalated treatment. In some embodiments, a classifier model is trained to determine whether an individual should receive an escalated treatment.

In some embodiments, collections of individuals with breast cancer that have had their biomolecules measured and/or infiltrating immune cell data assessed from a tumor biopsy at baseline and after initiation of targeted treatment such that dynamic measurements can be utilized to train a model to predict pCR. As detailed in the attached manuscript, both static biomolecule expression measurements and/or infiltrating immune cell data after initiation of targeted treatment and dynamic biomolecule expression measurements from baseline to after initiation of targeted treatment each provide a significant prediction of pCR and can be utilized as features in a regression model. Additional features can also be utilized in a regression model, including (but not limited to) treatment type, ER-status, PAM50 status, tumor size, tumor grade, cancer stage, age of patient, and patient ethnicity.

In a number of embodiments, a classifier model can be trained to classify pCR based on a set of one or more biomolecule expression measurements and/or infiltrating immune cell data. Biomolecule expression measurements and/or infiltrating immune cell data include (but are not limited to) expression level and/or infiltration data of a single region, average expression across multiple regions, summed expression across multiple regions, median expression across multiple regions, standard error expression across multiple regions, and standard deviation expression across multiple regions. In various embodiments, a classifier model utilizes HER2 signaling pathway biomolecules, epithelial tumor biomolecules, immune response and activation biomolecules, cell survival biomolecules, infiltrating immune cell data, or a combination thereof. Accordingly, a classifier model can utilize a set of one more measurements of the following biomolecules: HER2, AKT/p-AKT, S6/p-S6, PTEN, p-ERK, p-STAT3, PanCK, Ki67, Beta-catenin, CD45, CD3, CD4, CD8, CD27, CD44, CD45RO, OX40L, ICOS, Granzyme B, CD19, CD11c, CD163, CD68, CD56, CD66B, CD14, STING, PD1/PDL1, B7-H3, B7-H4, IDO-1, Lag3, VISTA, Beta-2 microglobulin and Bcl-2. Likewise, a model can utilize infiltrating immune cell data as determined by H&E staining or immunostaining.

In some embodiments, a classifier model utilizes a set of one or more biomolecule expression measurements, the set including expression of HER2. In some embodiments, a classifier model utilizes a set of one or more biomolecule expression measurements, the set including expression of Ki67. In some embodiments, a classifier model utilizes a set of one or more biomolecule expression measurements, the set including expression of pS6. In some embodiments, a classifier model utilizes a set of one or more biomolecule expression measurements, the set including expression of CD45. In some embodiments, a classifier model utilizes a set of one or more biomolecule expression measurements, the set including expression of CD56. In some embodiments, a classifier model utilizes a set of one or more biomolecule expression measurements, the set including expression of STING. In some embodiments, a classifier model utilizes a set of one or more biomolecule expression measurements, the set including expression of VISTA. In some embodiments, a classifier model utilizes a set of one or more biomolecule expression measurements, the set including expression of CD66B.

In some embodiments, a classifier model utilizes a set of two or more biomolecule expression measurements, the set including expression of HER2 and Ki67. In some embodiments, a classifier model utilizes a set of two or more biomolecule expression measurements, the set including expression of HER2 and pS6. In some embodiments, a classifier model utilizes a set of two or more biomolecule expression measurements, the set including expression of HER2 and CD45. In some embodiments, a classifier model utilizes a set of two or more biomolecule expression measurements, the set including expression of HER2 and CD56. In some embodiments, a classifier model utilizes a set of two or more biomolecule expression measurements, the set including expression of HER2 and STING. In some embodiments, a classifier model utilizes a set of two or more biomolecule expression measurements, the set including expression of HER2 and VISTA. In some embodiments, a classifier model utilizes a set of two or more biomolecule expression measurements, the set including expression of HER2 and CD66B.

In some embodiments, a classifier model utilizes a set of two or more biomolecule expression measurements, the set including expression of CD45 and HER2. In some embodiments, a classifier model utilizes a set of two or more biomolecule expression measurements, the set including expression of CD45 and Ki67. In some embodiments, a classifier model utilizes a set of two or more biomolecule expression measurements, the set including expression of CD45 and pS6. In some embodiments, a classifier model utilizes a set of two or more biomolecule expression measurements, the set including expression of CD45 and CD56. In some embodiments, a classifier model utilizes a set of two or more biomolecule expression measurements, the set including expression of CD45 and STING. In some embodiments, a classifier model utilizes a set of two or more biomolecule expression measurements, the set including expression of CD45 and VISTA. In some embodiments, a classifier model utilizes a set of two or more biomolecule expression measurements, the set including expression of CD45 and CD66B.

In some embodiments, a classifier model utilizes a set of three or more biomolecule expression measurements, the set including expression of HER2, CD45 and Ki67. In some embodiments, a classifier model utilizes a set of three or more biomolecule expression measurements, the set including expression of HER2, CD45 and pS6. In some embodiments, a classifier model utilizes a set of three or more biomolecule expression measurements, the set including expression of HER2, CD45 and CD56. In some embodiments, a classifier model utilizes a set of three or more biomolecule expression measurements, the set including expression of HER2, CD45 and STING. In some embodiments, a classifier model utilizes a set of three or more biomolecule expression measurements, the set including expression of HER2, CD45 and VISTA. In some embodiments, a classifier model utilizes a set of three or more biomolecule expression measurements, the set including expression of HER2, CD45 and CD66B.

In some embodiments, a classifier model utilizes a set of three or more biomolecule expression measurements, the set including expression of CD45, CD56 and HER2. In some embodiments, a classifier model utilizes a set of three or more biomolecule expression measurements, the set including expression of CD45, CD56 and Ki67. In some embodiments, a classifier model utilizes a set of three or more biomolecule expression measurements, the set including expression of CD45, CD56 and pS6. In some embodiments, a classifier model utilizes a set of three or more biomolecule expression measurements, the set including expression of CD45, CD56 and STING. In some embodiments, a classifier model utilizes a set of three or more biomolecule expression measurements, the set including expression of CD45, CD56 and VISTA. In some embodiments, a classifier model utilizes a set of three or more biomolecule expression measurements, the set including expression of CD45, CD56 and CD66B.

In some embodiments, a classifier model utilizes quantification of infiltrating immune cells. In some embodiments, a classifier model utilizes quantification of sTILs. In some embodiments, a classifier model utilizes infiltrate grade score of iTu-Ly. In some embodiments, a classifier model utilizes quantification CD45+ cells. In some embodiments, a classifier model utilizes quantification CD56+ cells.

In several embodiments, a classifier's sensitivity, specificity, and area under the curve (AUC) metrics can be modified to achieve desired performance. In some instances, higher specificity may be desired to ensure robust classification of individuals to ensure each individual is treated properly. In some instances, higher sensitivity is desired such that the limit-of-detection is lower, decreasing the number of missed true positive results. Accordingly, in various embodiments, specificity is set at about: 65%, 70%, 75%, 80%, 85%, 90%, 95%, 98%, 100%, or there between. And in various embodiments, sensitivity is set at about: 60%, 65%, 70%, 75%, 80%, 85%,90%, 95%, 98%, 100%, or there between.

Based upon a cancer's classification, a HER2+ breast cancer is treated 105 accordingly. In several embodiments, when a pCR is indicated, a deescalated treatment regimen is administered, such as (for example) a targeted treatment regimen directed at HER2 without generalized chemotherapy (i.e., non-targeted chemotherapy). Targeted treatments include (but not limited to) trastuzumab, lapatinib, pertuzumab, T-DM1, and any combination thereof. In some embodiments, a targeted chemotherapeutic agent is used (e.g., T-DM1). In many embodiments, when pCR is not indicated, an escalated treatment regimen is administered, such as (for example) a targeted treatment with chemotherapy regimen or dual-targeted therapy regimen (i.e., two targeted therapeutics). Chemotherapeutics include (but not limited to) taxanes including paclitaxel (Taxol), anthracyclines including doxorubicin (Adriamycin), cyclophosphamide, and any combination thereof.

While specific examples of processes for molecularly classifying and treating a breast cancer are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for molecularly classifying and treating appropriate to the requirements of a given application can be utilized in accordance with various embodiments.

### Methods of Measuring Biomolecule Expression

Biomolecule expression can be detected and measured by a number of methods in accordance with various embodiments, as would be understood by those skilled in the art. In several embodiments, breast cancer tumors are biopsied or surgically resected from a patient, fixed and prepared for detection and measurement of biomolecule expression. Any appropriate fixation method can be utilized, including (but not limited to) formaldehyde, formalin fixed paraffin embedded (FFPE), methanol, ethanol, OCT embedding, and flash freezing.

It has been found that detecting and measuring biomolecules in regions of interest of the tumor can provide a better prediction of pCR than bulk RNA profiling of the tumor. Accordingly, in several embodiments, regions of interest or particular cell types are identified and used for biomolecule detection and measurement techniques. In some embodiments, tissue is treated with an antibody and/or stained such that regions of interest can be identified via microscopy in which detection and measurement of biomolecules can be performed directly on the regions of interest. In some embodiments, regions of interest are identified by panCK+ tumor cells. In some embodiments, regions of interest are identified by CD45+ immune cells. In some embodiments, multiplex spatial tissue analysis is performed to determine biomolecule expression. In some embodiments, live or fixed tissue is treated with an antibody and/or stained such that cell types can be identified and isolated via flow cytometry in which the isolated cells can be used to extract biomolecules for detection and measurement.

In many embodiments, multiplex spatial tissue analysis is utilized to detect protein and/or RNA expression in regions of interest of fixed tissue. In some embodiments, protein and RNA expression is simultaneously assessed in regions of interest of fixed tissue. There are a number of methodologies and kits to perform multiplex spatial tissue analysis, including (but not limited to) NanoString's GeoMx™ Digital Spatial Profiler (DSP) (Seattle, Wash.), Akoya Biosciences' CODEX (Menlo Park, Calif.), Akoya Biosciences' Vectra Polaris, Harvard Program in Therapeutic Science's Cyclic Immunofluorescence (CyCIF) (Boston, Mass.), IonPath's Multiplexed Ion Bea Imaging (MIBI) (Menlo Park, Calif.), Akoya Biosciences Opal kit, Roche-Ventana's DISCOVERY system (Oro Valley, Az.), and Genotipix-HistoRx's Automated Quantitative Analysis (AQUA) (New Haven, Conn.). In general, these systems can detect multiple biomolecules within regions of interest. Further review of protocols to analyze tissue can be found within E. R. Parra J. Cancer Treat. Diagn. 2018, 2, 43-53, and E. R. Parra, A. Ffancisco-Cruz, and I. I. Wistuba Cancers (Basel). 2019, 11, E247, the disclosures of which are each incorporated herein by reference.

One example of multiplex spatial tissue analysis is NanoString's GeoMx™ Digital Spatial Profiler (DSP), which can detect expression of RNA or peptides in a selected region, utilizing panels of oligos for RNA expression and/or antibodies for peptide expression. The details of this machine and methods to utilize this machine are described within the Exemplary Embodiments. In general, an identified region of interest (e.g., panCK+ region) is selected and the panels of antibodies and/or probes are incubated in the region of interest to bind and identify biomolecules of interest. After incubation, excess and unbound reagents are then washed away. Each antibody and probe within the panel has an attached oligo tail that is used as a barcode. The oligo tail barcode is releasable by UV irradiation. After biomolecule attachment and wash, UV light releases the barcodes which are then detected and measured using the NanoString nCounter, which determines the relative concentration (normalized to controls) of biomolecules of interest.

In embodiments in which extraction of biomolecules is performed, several methods are well known to extract biomolecules from biological sources. Generally, biomolecules are extracted from cells or tissue, then prepped for further analysis. Alternatively, biomolecules can be observed within cells, which are typically fixed and prepped for further analysis. The decision to extract biomolecules or fix tissue for direct examination depends on the assay to be performed. In general, in situ hybridization and histology samples are performed in fixed tissues, whereas nucleic acid proliferation techniques (e.g., sequencing) and protein quantification techniques (e.g., ELISA) are performed utilizing extracted biomolecules.

In several embodiments, cells utilized to examine biomolecules are neoplastic cells of a breast cancer and/or infiltrating immune cells, which can be extracted or analyzed directly in a biopsy. In some embodiments, a solid tumor biopsy is utilized, such as (for example) a primary, nodal, and/or distal tumor. In some embodiments, regions of interest are determined by detecting tumor cells (e.g., pancytokeratin-positive (panCK+) tumor cells), infiltrating immune cells (e.g., CD45-positive (CD45+)), or a combination thereof. It is to be understood that any appropriate means or biomarkers to identify regions of interest or isolate particular cell types can be utilized in accordance with various embodiments.

A number of assays are known to determine biomolecule expression in a biological samples, including (but not limited to) hybridization techniques, nucleic acid proliferation techniques, sequencing, antibody detection, and mass spectrometry. A number of hybridization techniques can be used, including (but not limited to) in situ hybridization, microarrays (e.g., Affymetrix, Santa Clara, Calif.), and NanoString nCounter (Seattle, Wash.). Likewise, a number of nucleic acid proliferation techniques can be used, including (but not limited to) PCR and RT-PCR. In addition, a number of sequencing techniques can be used, including (but not limited to) genome sequencing, exome sequencing, targeted gene sequencing, Sanger sequencing, and RNA-seq of tumor tissue. A number of antibody techniques can be used, including (but not limited to) in situ histology/immunohistochemistry, immunofluorescence staining and cyclic immunofluorescence staining, ELISA, and Western blot.

As understood in the art, only a portion of a genomic locus, gene, or peptide may need to be detected in order to have a positive detection. In many hybridization techniques, detection probes are typically between ten and fifty bases, however, the precise length will depend on assay conditions and preferences of the assay developer. In many amplification techniques, amplicons are often between fifty and one-thousand bases, which will also depend on assay conditions and preferences of the assay developer. In many sequencing techniques, genomic loci and transcripts are identified with sequence reads between ten and several hundred bases, which again will depend on assay conditions and preferences of the assay developer. In many antibody techniques, monoclonal or polyclonal antibodies may be used. In some embodiments, hybridization, targeted sequencing, and antibody detection techniques are directed to sequences of a number of genes of interest, such as those that confer an indication of pCR of a breast cancer.

It should be understood that minor variations in gene sequence and/or assay tools (e.g., hybridization probes, amplification primers) may exist but would be expected to provide similar results in a detection assay. These minor variations are to include (but not limited to) insertions, deletions, single nucleotide polymorphisms, and other variations due to assay design. In some embodiments, detection assays are able to detect genomic loci and transcripts having high homology but not perfect homology (e.g., 70%, 80%, 90%, 95%, or 99% homology). In some embodiments, detection assays are able to detect genomic loci and transcripts having 1 base pair changed, deleted or inserted, 2 base pairs changed, deleted or inserted, 3 base pairs changed, deleted or inserted, 4 base pairs changed, deleted or inserted, 5 base pairs changed, deleted or inserted, or more than 5 base pairs changed, deleted or inserted. As understood in the art, the longer the nucleic acid polymers used for hybridization, less homology is needed for the hybridization to occur.

It should also be understood that several gene transcripts have a number isoforms that are expressed. As understood in the art, many alternative isoforms would be understood to confer similar indication of molecular classification, and thus metastatic potential. Accordingly, alternative isoforms of gene transcripts are also covered in some embodiments.

### Assessment of Infiltrating Immune Cells

Infiltrating immune cells can be detected and assessed by a number of methods in accordance with various embodiments, as would be understood by those skilled in the art. In several embodiments, breast cancer tumors are biopsied or surgically resected from a patient, fixed and prepared for detection and assessment of immune cell infiltration. Any appropriate fixation method can be utilized, including (but not limited to) formaldehyde, formalin fixed paraffin embedded (FFPE), methanol, ethanol, OCT embedding, and flash freezing.

It has been found that detecting and assessing infiltrating immune cells in regions of interest of the tumor can provide robust prediction of pCR. Accordingly, in several embodiments, regions of interest or particular cell types are identified and used for infiltrating immune cell detection and assessment techniques. In some embodiments, tissue is treated with an antibody and/or stained such that regions of interest can be identified via microscopy in which detection and assessment of infiltrating immune cells can be performed directly on the regions of interest. In some embodiments, regions of interest are identified by panCK+ tumor cells. In some embodiments, regions of interest are identified by CD45+ immune cells.

In many embodiments, histological analysis is performed by histological staining and/or immune staining. In some embodiments, cancer biopsies can be stained with hematoxylin and eosin (H&E) and infiltrating immune cells can be counted. In some embodiments, H&E stained cancer biopsies are assessed to quantify infiltration of stromal tumor infiltrating lymphocytes (sTILs) or intratumoral lymphocytes (iTu-Ly). Typically, sTILs are quantified as a score of 0-100% as determined by the percent of sTILs of total cells in a region of interest. iTu-Ly is typically scored via a semi-quantitative infiltrate grade (0 to 3). In some embodiments, cancer biopsies can be assessed by immunostaining with an anti-CD45 antibody and/or an anti-CD56 to determine the number of infiltrating lymphocytes. Immunostaining can be performed in a number ways, including (but not limited to) chromogenic immunohistochemistry (IHC), immunofluorescence, or elemental isotope staining (e.g., antibodies labeled elemental isotopes). Infiltrating lymphocytes can be quantified in a number of ways, typically as percentage. In some embodiments, infiltrating lymphocytes are quantified as a percentage of total cells in a region of interest. In some embodiments, infiltrating lymphocytes are quantified as a percentage of total lymphocytes (e.g., number of lymphocytes in tumor tissue divided by total number of lymphocytes in tumor and surrounding tissues). In some embodiments, infiltrating lymphocytes are quantified as a number of counts per area (e.g., mm²). In various embodiments, histological analysis is performed by a pathologist and/or automated image analysis machine. For more on histological analysis of infiltrating immune cells, see R. Salgado, et al., Ann Oncol. 2015 26(2):259-71; and C. Denkert, et al., Mod Pathol. 2016 October; 29(10):1155-64; the disclosures of which are each incorporated herein by reference.

### Kits

In several embodiments, kits are utilized for determining whether a breast cancer is likely to achieve a pCR after targeted treatment. Kits can be used to detect expression of biomarkers in regions of interest of a biopsy as described herein. For example, the kits can be used to detect any one or more of the gene biomarkers described herein, which can be used to determine likelihood of a pCR. The kit may include one or more agents for determining biomolecule expression, one or more agents for assessing infiltration of immune cells, a container for collecting a biological sample (e.g., biopsy) obtained from a subject, appropriate means for fixing and preparing the biological sample (e.g., reagents and materials for FFPE), and reagents to identifying regions of interest, and printed instructions for reacting agents with the biological sample to detect expression of biomarker genes derived from the sample. The agents may be packaged in separate containers. The kit may further comprise one or more control reference samples and reagents for performing a biochemical assay, enzymatic assay, immunoassay, hybridization assay, or sequencing assay.

In several embodiments, kits are used to detect and measure biomolecules of interest. A nucleic acid detection kit, in accordance with various embodiments, includes a set of hybridization-capable complement sequences and/or amplification primers specific for a set of genomic loci and/or expressed transcripts. In some instances, a kit will include further reagents sufficient to facilitate detection and/or quantitation of a set of genomic loci and/or expressed transcripts. In some instances, a kit will be able to detect and/or quantify expression for at least 5, 10, 15, 20, 25, 30, 40 or 50 biomolecules. In some instances, a kit will be able to detect and/or quantify expression of thousands or more biomolecules via a sequencing technique.

In a number of embodiments, a set of hybridization-capable complement sequences are immobilized on an array, such as those designed by Affymetrix or IIlumina. In many embodiments, a set of hybridization-capable complement sequences are linked to a “bar code” to promote detection of hybridized species and provided such that hybridization can be performed in solution, such as those designed by NanoString. In several embodiments, a set of primers (and, in some cases probes) to promote amplification and detection of amplified species are provided such that a PCR can be performed in solution, such as those designed by Applied Biosystems of ThermoScientific (Foster City, Calif.).

A kit can include one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of determining biomolecule expression of a tumor biopsy.

### Applications and Treatments for HER2+ Breast Cancer

Various embodiments are directed to breast cancer diagnostics and treatments based on an indication of whether the cancer is likely to achieve a pCR after targeted treatment, especially short-term targeted treatment. As described herein, a prognostic procedure can utilize regions of interest of a biopsy to detect and determine biomolecule expression and/or immune cell activation, especially biomolecules related to HER2+ signaling and immune response and activation. Biomolecule expression and/or immune cell activation and a trained classifier is used to classify a breast cancer into likely to achieve pCR not likely to achieve a pCR by targeted treatment alone. Based on the likelihood to achieve a pCR, appropriate treatments to the individual can be administered.

### Diagnostic Indication and Treatments

A number of embodiments are directed towards getting a diagnostic indication of how to treat a breast cancer after initiation of a targeted treatment. In some embodiments, a cancer biopsy is extracted after initiation of targeted treatment from the individual that has the breast cancer and the biopsy is further analyzed.

In a number of embodiments, a diagnostic indication can be performed on a breast cancer patient as follows:
a) perform at least one cycle of targeted treatment
b) extract a biopsy
c) determine static and/or dynamic expression of a set of one or more biomarkers
d) diagnose whether targeted treatment alone can provide a pCR and determine an appropriate treatment strategy

In accordance with several embodiments, once an indication of whether a breast cancer can achieve a pCR with targeted treatment, a deescalated treatment is administered. In several embodiments, when a pCR is indicated for a breast cancer, a targeted treatment is administered without generalized chemotherapy (i.e., nontargeted chemotherapy), especially in the neoadjuvant setting. In some embodiments, the breast cancer is HER2+ and the targeted treatment targets HER2. In some embodiments, a targeted chemotherapeutic agent is used (e.g., T-DM1). Targeted HER2 treatments include (but not limited to) trastuzumab, lapatinib, pertuzumab, T-DM1, and any combination thereof. In many embodiments, when pCR is not indicated for a breast cancer, an escalated treatment is administered in the neoadjuvant and/or adjuvant settings. In some embodiments, when pCR is not indicated, a targeted treatment with chemotherapy is administered. In some embodiments, when pCR is not indicated, a dual-targeted treatment with chemotherapy is administered. Chemotherapeutics include (but not limited to) taxanes including paclitaxel (Taxol), anthracyclines including doxorubicin (Adriamycin), cyclophosphamide, and any combination thereof.

In some embodiments, a diagnosis is determined based on threshold. In some embodiments, a threshold is determined by a classifier's sensitivity, specificity, and/or area under the curve (AUC) metrics. In some instances, a threshold with a higher specificity may be desired to ensure robust classification of individuals to ensure each individual is treated properly. For instance, it may be desirable to have high specificity when classifying individuals as likely to achieve pCR. If an individual is misclassified as likely to achieve pCR but instead as fails to achieve pCR from neoadjuvant treatment, treatment regimens may require harsher chemotherapeutics and/or to be prolonged and thus the individual would have been better off receiving a targeted treatment with chemotherapy initially. In some instances, higher sensitivity is desired such that the limit-of-detection is lower, decreasing the number of missed true positive results. Accordingly, in various embodiments, specificity is set at about: 65%, 70%, 75%, 80%, 85%, 90%, 95%, 98%, 100%, or there between. And in various embodiments, sensitivity is set at about: 60%, 65%, 70%, 75%, 80%, 85%, 90%, 95%, 98%, 100%, or there between.

Specific treatment regimens are also contemplated. In some embodiments, when a pCR is indicated for a HER2+ breast cancer, the following combinations of therapeutics are administered in a treatment regimen:
trastuzumab and lapatinib
trastuzumab and pertuzumab
T-DM1 alone
T-DM1 and pertuzumab

In some embodiments, when a pCR is not indicated for a HER2+ breast cancer, the following combinations of therapeutics are administered in a treatment regimen:
trastuzumab, pertuzumab, and a chemotherapeutic
T-DM1 and pertuzumab, followed by weekly paclitaxel, doxorubicin, and cyclophosphamide
trastuzumab, pertuzumab, and a taxane
T-DM1, pertuzumab, and an anthracycline (e.g., doxorubicin)

It should be understood that the specific therapeutic combinations should not be considered limiting and that a number of combinations of targeted therapeutics and/or chemotherapeutics can be administered.

Dosing and therapeutic regimes can be administered appropriate to the breast cancer to be treated, as understood by those skilled in the art. For example, the following dosing amounts can be utilized in a treatment cycle in accordance with various embodiments:
Pertuzumab: 840 mg IV infusion over 60 min, then 420 mg IV infusion over 30-60 min plus
Trastuzumab: 8 mg/kg IV infusion over 90 min initially, then 6 mg/kg IV infusion over 30-90 min plus
Paclitaxel: 175 mg/m² IV infusion over 3 hours
Doxorubicin: 60 mg/m² IV infusion

In some embodiments, medications are administered in a therapeutically effective amount as part of a course of treatment. As used in this context, to “treat” means to ameliorate at least one symptom of the disorder to be treated or to provide a beneficial physiological effect. For example, one such amelioration of a symptom could be reduction of tumor size and/or achieving pCR.

A therapeutically effective amount can be an amount sufficient to prevent reduce, ameliorate or eliminate the symptoms of breast cancer. In some embodiments, a therapeutically effective amount is an amount sufficient to reduce the growth and/or metastasis of a breast cancer. In some embodiments, a therapeutically effective amount is an amount sufficient to achieve pCR.

### EXEMPLARY EMBODIMENTS

The embodiments of the invention will be better understood with the several examples provided within. Many exemplary results of processes that identify combinatorial molecular indicators of colorectal cancer are described. Validation results are also provided.

### Example 1

### Spatial Proteomic Characterization of HER2-Positive Breast Tumors through Neoadjuvant Therapy Predicts Response

Human epidermal growth factor receptor 2 (HER2)-positive breast cancer accounts for 15-30% of invasive breast cancers and is associated with an aggressive phenotype. While the addition of HER2-targeted agents to neoadjuvant chemotherapy has dramatically improved pathological complete response (pCR) rates in early stage HER2-positive breast cancer, 40-50% of patients have residual disease after treatment. Conversely, HER2 inhibition with two targeted agents and without chemotherapy can result in pCR, suggesting that it may be possible to eliminate chemotherapy in a subset of patients. Identification of biomarkers that provide an indication of response to HER2-targeted therapy can help delineate the regimen of neoadjuvant therapy.

Bulk gene expression profiling of pre-treatment samples has identified tumor characteristics (HER2-enriched intrinsic subtype, HER2 expression levels, and ESR1 expression levels), and microenvironmental characteristics (increased immune infiltration) that associate with response to HER2-targeted therapy in the neoadjuvant setting. Because tumor cells are profiled simultaneously with both co-localized and distant stroma and immune cells, bulk expression profiling is an imperfect tool for analyzing tumor and microenvironmental changes across treatment. In particular, it is difficult to assign observed changes to specific geographic or phenotypic cell populations within the complex tumor ecosystem, where malignant tumor cells interact with fibroblasts, endothelial cells, and immune cells. Moreover, immune cells can be further divided into those that infiltrate the tumor core and those that are excluded. As of yet, how the tumor and immune microenvironment change during therapy remains poorly understood, necessitating multiplexed in situ profiling of longitudinal tissue samples.

The GeoMx™ Digital Spatial Profiling (DSP, NanoString) technology was used to assay archival tissue from an initial discovery set of 28 patients with HER2-positive breast cancer enrolled on the neoadjuvant TRIO-US B07 clinical trial (S. Hurvitz, et al., medRxiv 2020.09.16.20194324 (2020), the disclosure of which is incorporated herein by reference), whose tumors were sampled pre-treatment, after 14-21 days of HER2-targeted therapy, consisting of lapatinib, trastuzumab, or both (on-treatment), and at the time of surgery after completion of combination chemotherapy with HER2-targeted therapy (post-treatment). The results were subsequently validated in an independent validation set of 29 patients from the B07 clinical trial. Importantly, the neoadjuvant setting allows for early assessment of treatment response and pCR is a strong surrogate for long-term survival in HER2-positive disease (J. Huober, et al., Eur J Cancer 118, 169-177 (2019); K R. Broglio, et al., JAMA Oncol 2, 751-760 (2016); and P. Cortazar, et al., Lancet 384, 164-172 (2014); the disclosures which are each incorporated herein by reference). DSP enables geographic and phenotypic selection of tissue regions for multiplex proteomic characterization of cancer signaling pathways and the tumor-colocalized immune microenvironment (M. I. Toki, et al., Cancer Research 77, 3810 (2017); and C. R. Merritt, et al., Nat Biotechnol 38, 586-599 (2020); the disclosures of which are each incorporated herein by reference). In particular, spatial heterogeneity was characterized in untreated breast tumors as well as changes in cancer signaling pathways and microenvironmental composition in matched on-treatment biopsies and post-treatment surgical samples by profiling 40 tumor and immune proteins across multiple pancytokeratin (panCK)-enriched regions per sample. On-treatment protein expression changed dramatically in tumors that went on to achieve a pCR and a classifier based on these data robustly predicted treatment response in the validation cohort. This new spatial-proteomic biomarker outperformed established predictors such as PAM50 subtype as well as classifiers based on transcriptomic data in this cohort, suggesting new avenues to personalize therapy in early-stage HER2-positive breast cancer.

### RESULTS

### Spatial Proteomic Analysis of Untreated HER2-Positive Breast Tumors

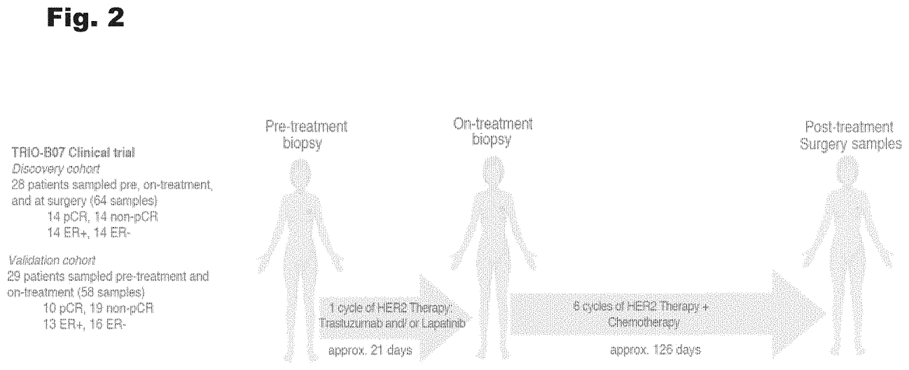

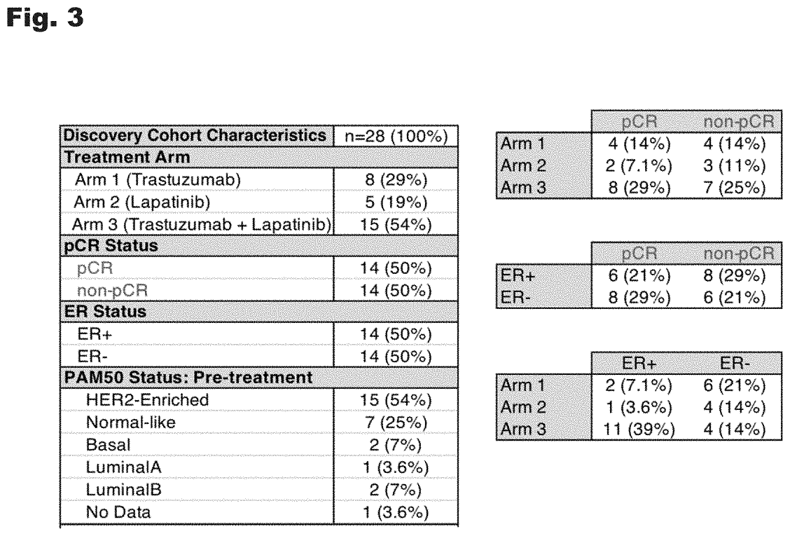

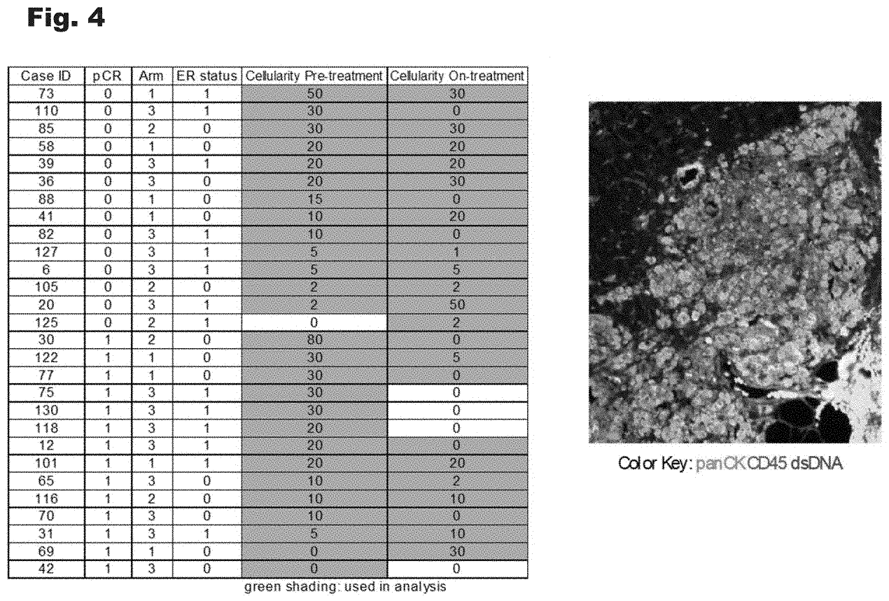

Participants in the TRIO-US B07 clinical trial (NCT00769470 in early-stage HER2-positive breast cancer) received one cycle of neoadjuvant HE R2-targeted therapy, including either trastuzumab, lapatinib, or both agents, followed by six cycles of the assigned HER2-targeted therapy plus docetaxel and carboplatin given every three weeks (S. Hurvitz, et al., (2020), cited supra). Core biopsies were obtained pre-treatment and on-treatment after 14-21 days of HER2-targeted therapy, and surgical resection specimens were obtained post-treatment (FIG. 2). Initially, a discovery cohort included 28 patients for whom FFPE samples were available from all three timepoints (pre-treatment, on-treatment, and at surgery). The cohort was balanced for both pCR and ER status (FIGS. 3 and 4) and was used for all exploratory analyses. A validation cohort of 29 patients from the TRIO-US B07 cohort with matched pre and on-treatment FFPE samples was utilized for evaluation of model performance.

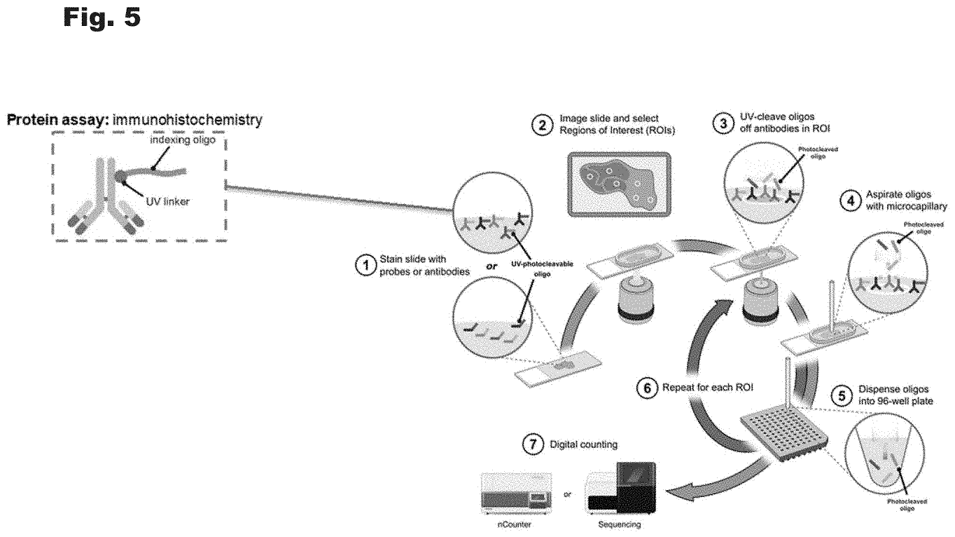

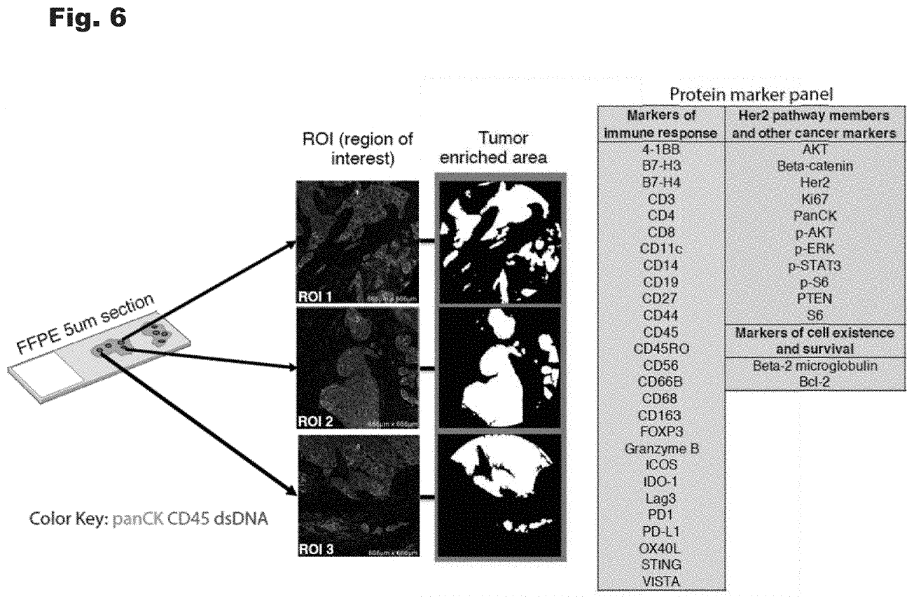

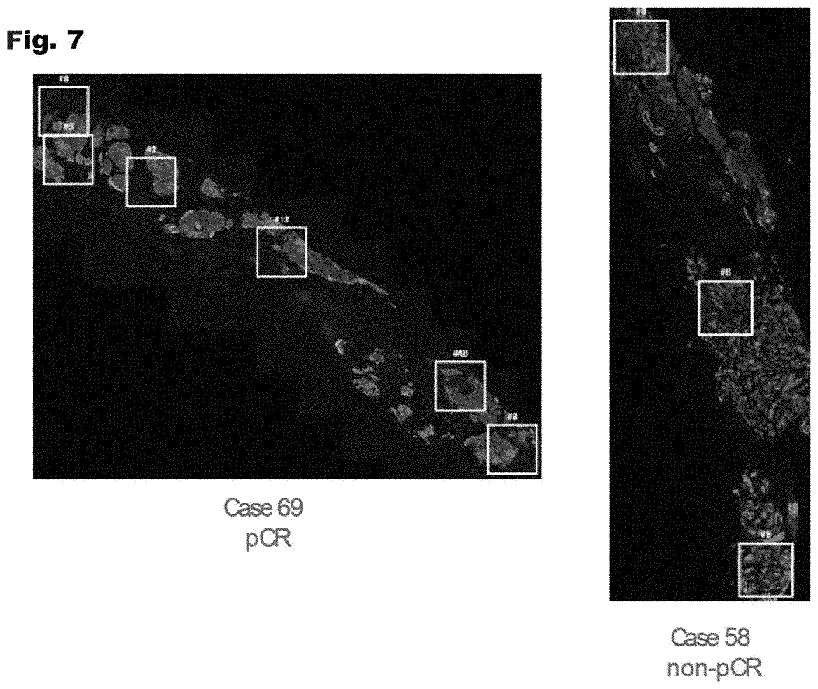

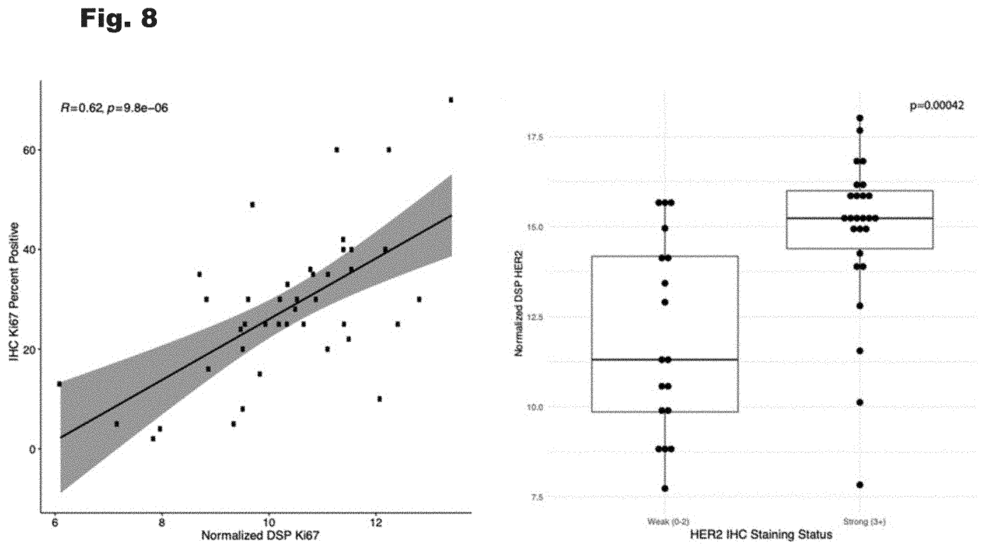

DSP enables multiplex proteomic profiling of formalin-fixed paraffin-embedded (FFPE) tissue sections (FIG. 5), where regions of interest (ROIs) can be selected based on both geographic and phenotypic characteristics. A panCK enrichment strategy was employed to profile cancer cells and colocalized immune cells across an average of four regions per tissue specimen (FIG. 6). Using CD45, panCK, and dsDNA were selected immunofluorescent markers for visualization, spatially separated regions (FIG. 7) and a mask governing the UV illumination for protein quantitation was generated based on panCK immunofluorescence. In total, 40 tumor and immune proteins were profiled using DSP, and proteins assessed using both DSP and orthogonal technologies showed strong concordance (FIGS. 6 and 8). Paired pre and on-treatment bulk gene expression data from the same patients was utilized to infer PAM50 subtype and enable comparisons with the spatially resolved DSP data.

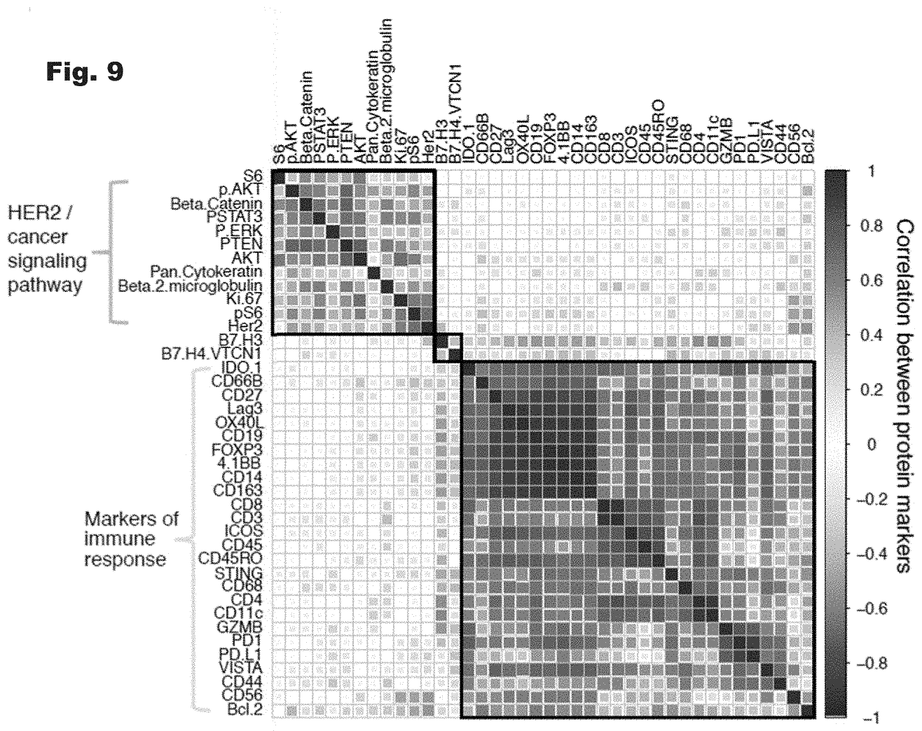

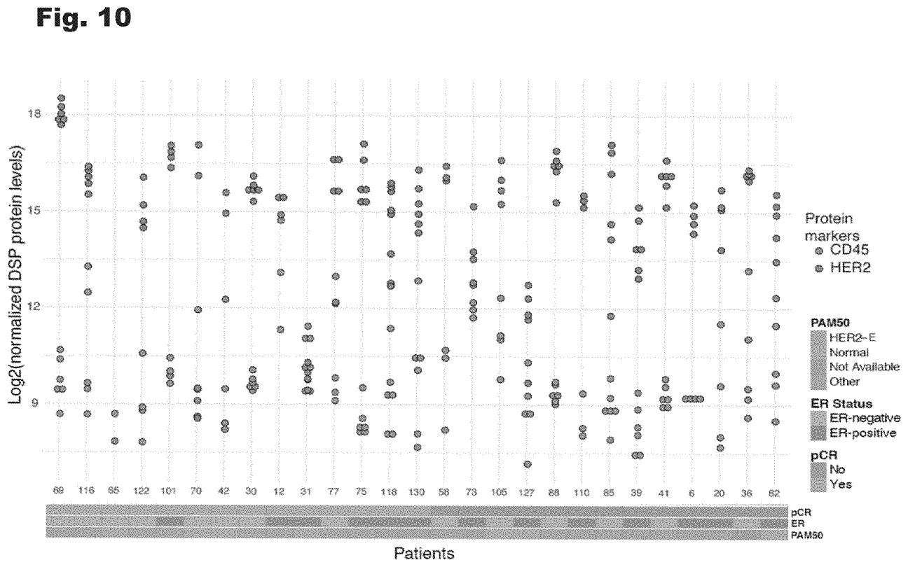

In untreated tumors, the correlation amongst immune markers was striking, suggesting the coordinated action of multiple immune cell subpopulations (FIG. 9). HER2 pathway members and other downstream cancer signaling markers were also highly correlated, while the correlation between tumor and immune markers was minimal for most marker pairs. Inter- and intra-tumor variability at the proteomic level was evident prior to treatment, including for HER2 and the pan-leukocyte marker CD45 (FIG. 10). Averaging all ROIs per patient to derive a composite score per marker, it was found that baseline HER2 levels were similar among tumors that achieved a pCR versus those that did not (mean pCR cases: 14.50, mean non-pCR cases: 15.00), as were CD45 levels (mean pCR: 9.90, mean non-pCR: 9.64). Using a linear mixed-effects model with blocking by patient (Methods), it was further found that individual DSP protein markers, including HER2 and CD45, did not significantly differ in pCR versus non-pCR cases prior to treatment (unadjusted p>0.10 for all markers).

### Decreased Cancer Signaling and Increased Immune Infiltration after Short-Term HER2-Targeted Therapy

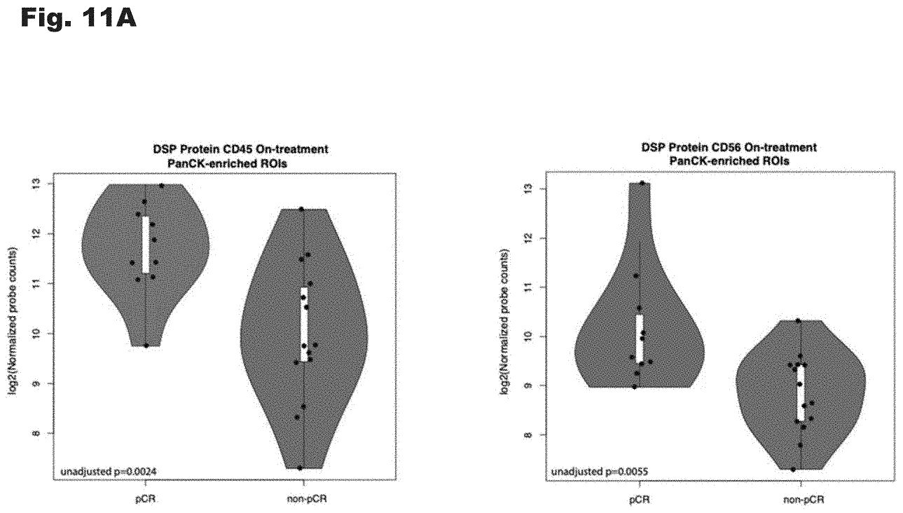

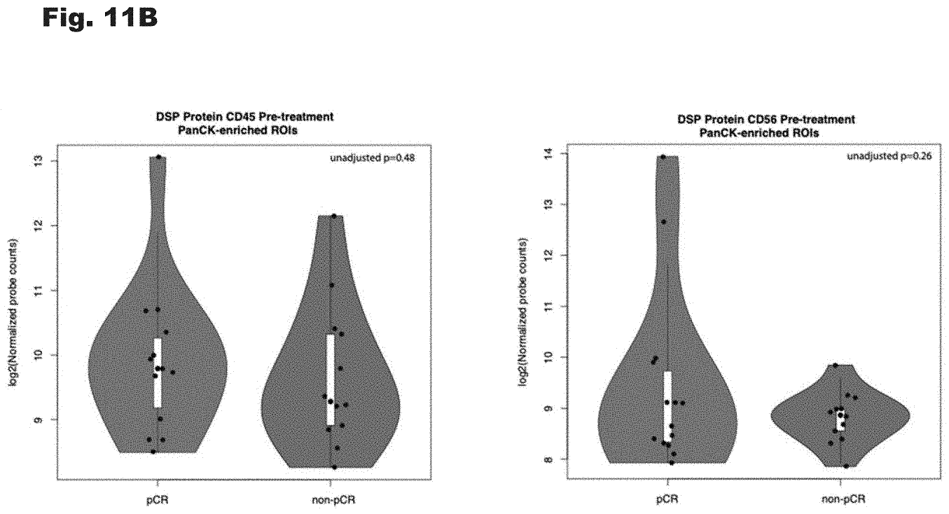

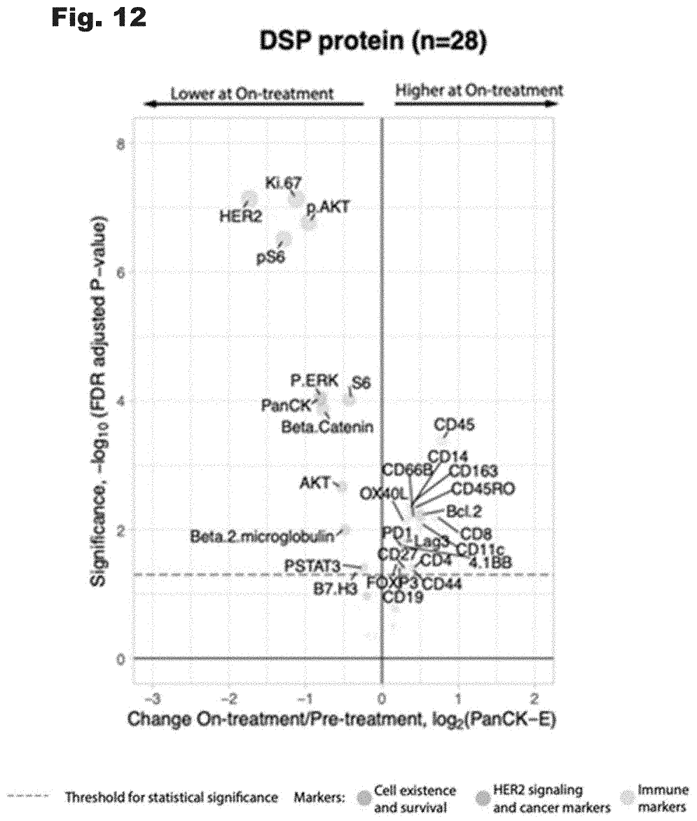

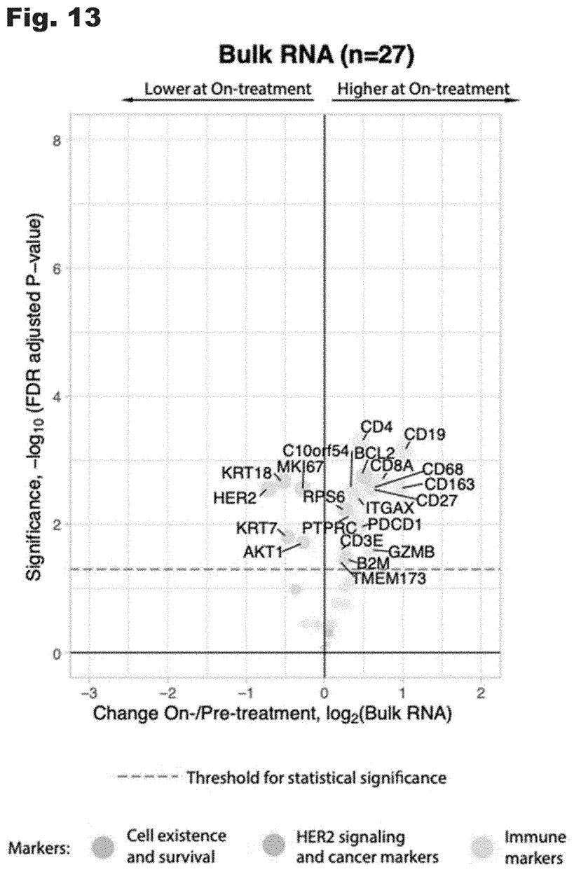

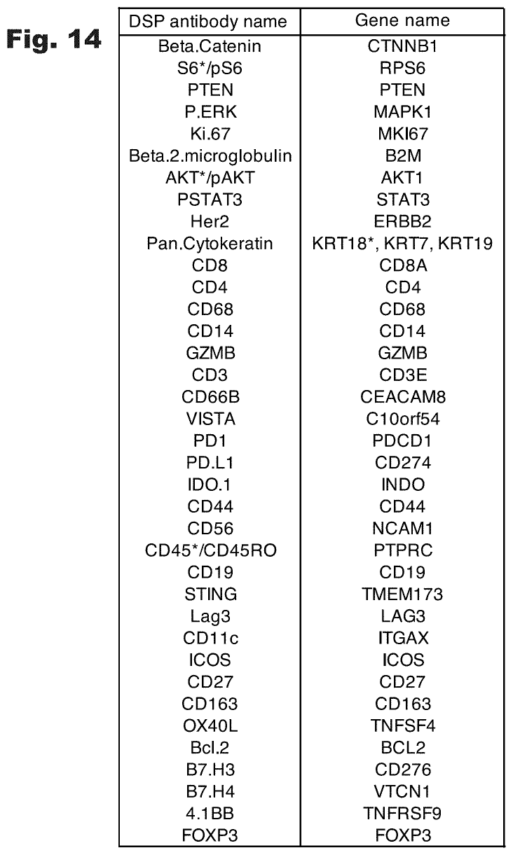

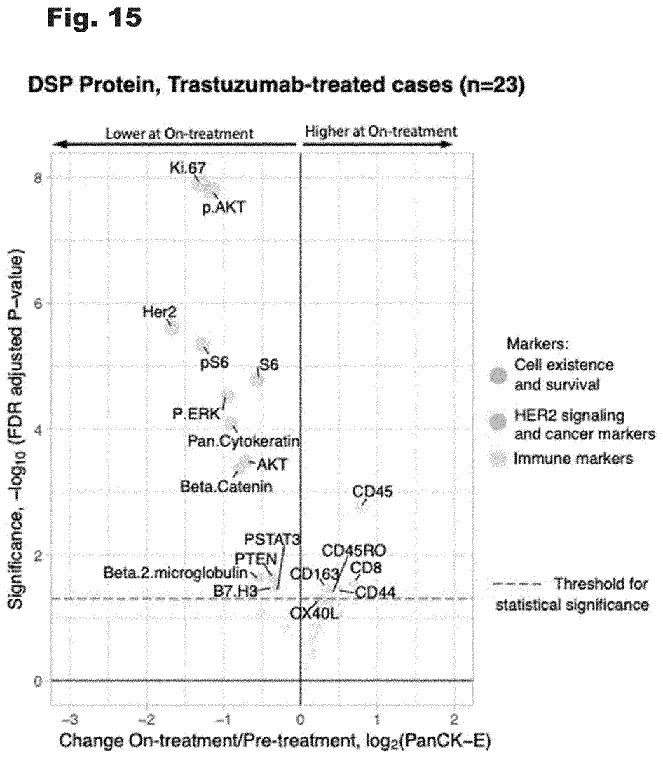

DSP was used to investigate treatment-related changes in both breast tumor and immune markers during short-term HER2-targeted therapy by profiling on-treatment (after a single cycle of HER2-targeted therapy alone) biopsies in the discovery cohort. The protein markers that were most associated with pCR at the on-treatment timepoint were CD45 (unadjusted p=0.0024) and CD56, a natural killer (NK) cell marker (unadjusted p=0.0055) (FIGS. 11A and 11B). The fold change in protein levels on-treatment relative to pre-treatment was quantified using a linear mixed-effects model with blocking by patient and visualized the significance (false discovery adjusted p-value) of all markers relative to their fold change in volcano plots. These analyses revealed a dramatic reduction in HER2 and Ki67, accompanied by other downstream pathway members, including pAKT, AKT, pERK, S6, and pS6, with the phosphorylated proteins decreasing comparatively more (FIG. 12). Immune markers—including CD45 and CD8, a marker of cytotoxic T-cells—exhibited the greatest increase in expression with treatment. Of note, increased expression of CD8+ T-cells was similarly observed in the TRIO-US B07 transcriptomic data through cell-type deconvolution, however given the lack of a control arm undergoing repeated biopsy without intervening treatment, it is uncertain whether the immune changes observed were related to HER2-targeted therapy or repeated biopsy. More generally, the on- versus pre-treatment bulk transcriptome data mirrored the changes seen at the protein level, but the fold changes were attenuated (FIG. 13). For example, using genes that correspond with the DSP protein markers (FIG. 14), it was found that the expression of HER2, AKT, Ki67, and breast cancer-associated keratin genes (KRT7, KRT18, and KRT19) decreased significantly with treatment, while immune markers increased. Despite the use of different analytes, measurements, and tissue sections, the DSP protein and bulk RNA datasets consistently showed decreased HER2 signaling and breast cancer-associated markers, accompanied by increased immune cell infiltration during neoadjuvant treatment. Given that lapatinib was associated with lower pCR rates in the TRIO-US B07 trial, on-treatment changes in the trastuzumab-treated cases (arms 1 and 3, n=23) were additionally assessed and similar patterns as in the full cohort were observed (FIG. 15).

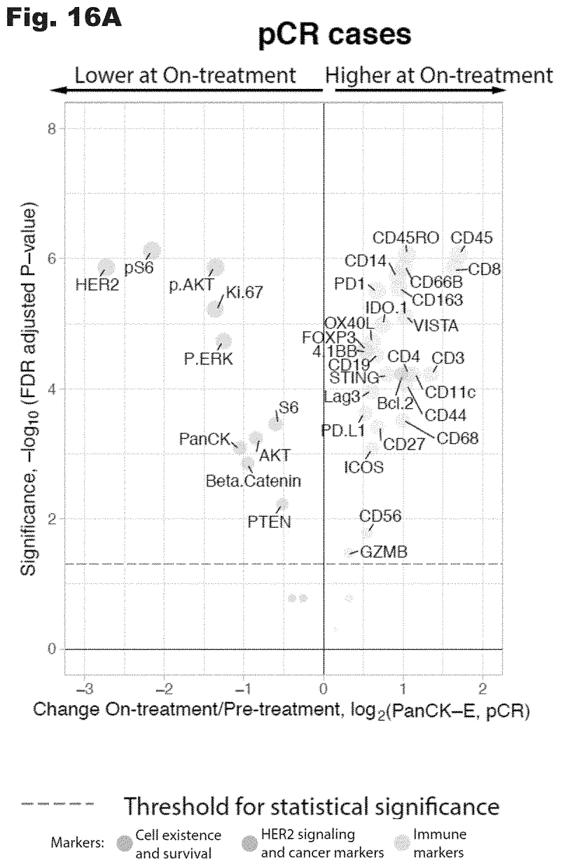

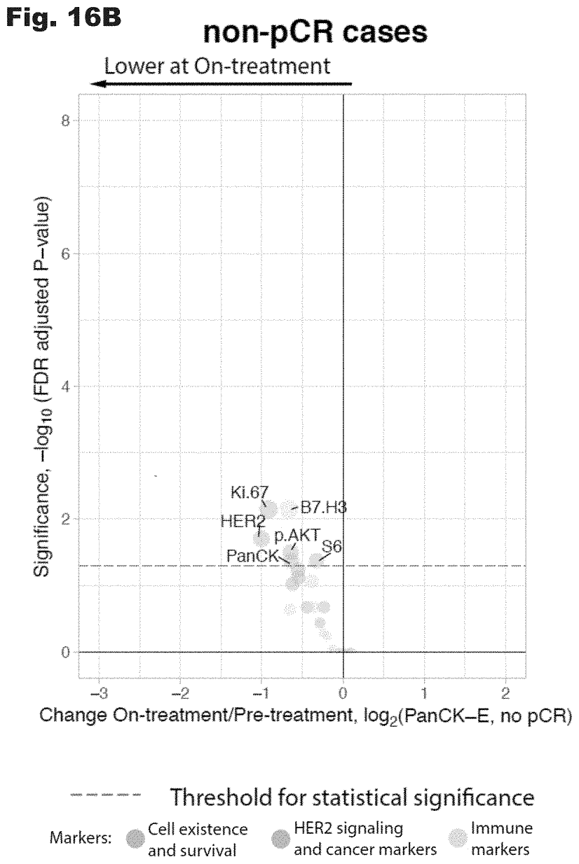

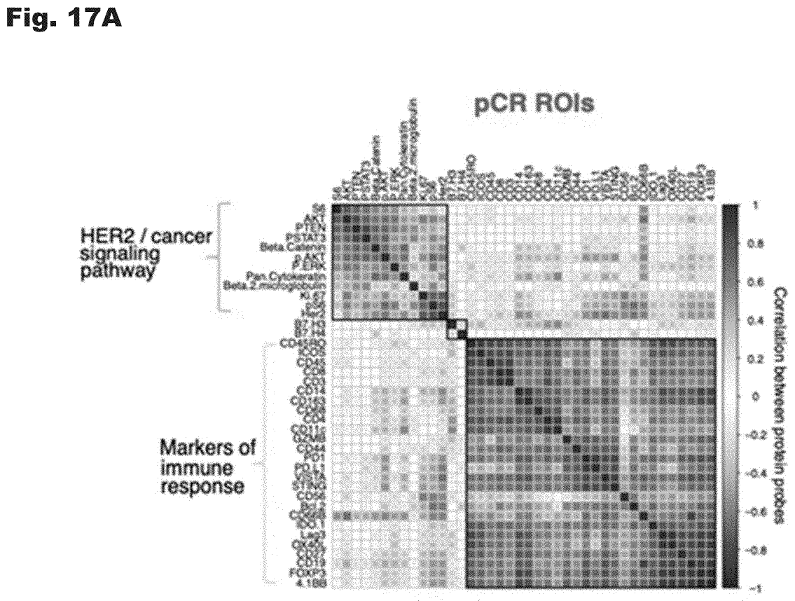

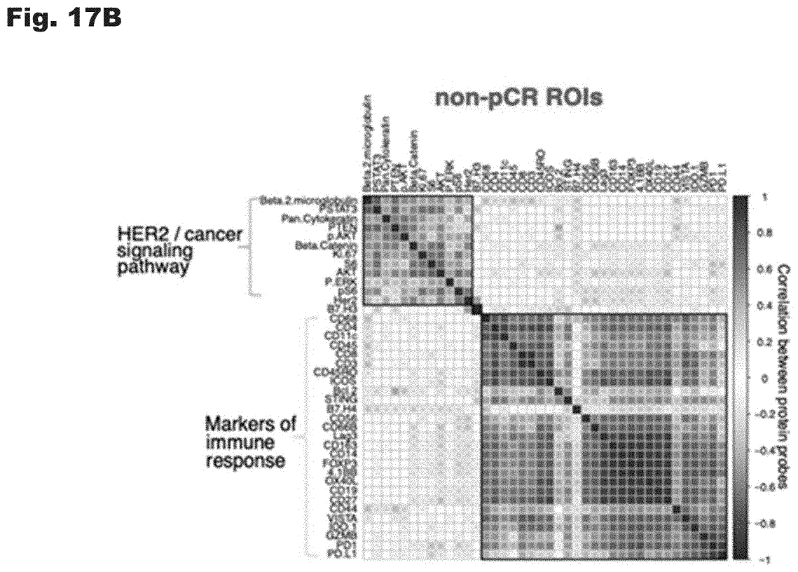

It was next examined how treatment-associated changes differed based on tumor sensitivity to HER2-targeted therapy, stratifying tumors based on achievement of pCR following neoadjuvant therapy (FIGS. 16A and 16B). In the pCR cases, a multitude of immune markers increased with treatment, while, in the non-pCR cases, no significant treatment-associated immune changes were observed and the reduction in Ki67 and HER2 signaling was modest. These patterns can also be visualized via pairwise comparisons of protein marker correlations, which revealed a stronger negative correlation between the immune marker cluster and the cancer cell marker cluster in tumors that went on to achieve a pCR (mean fold change across all markers in pCR cases: −0.231, non-pCR cases: −0.075, two-sided Wilcoxon rank sum test p<2.2e-16) (FIGS. 17A and 17B).

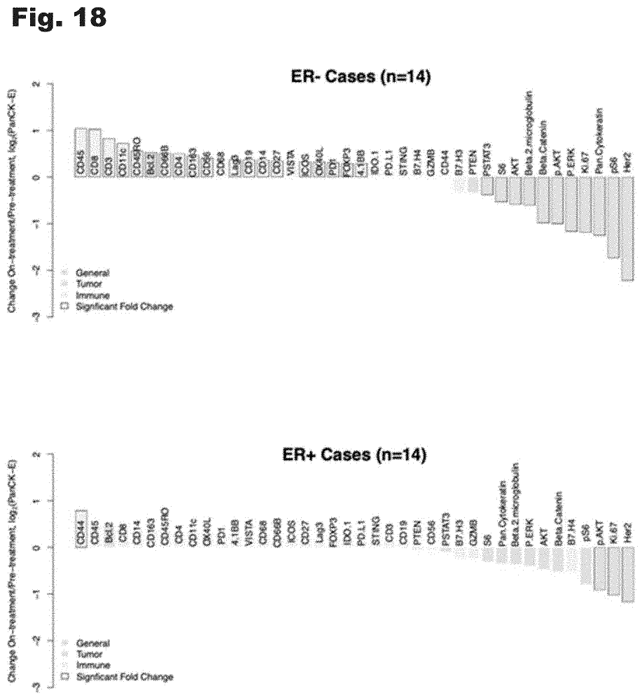

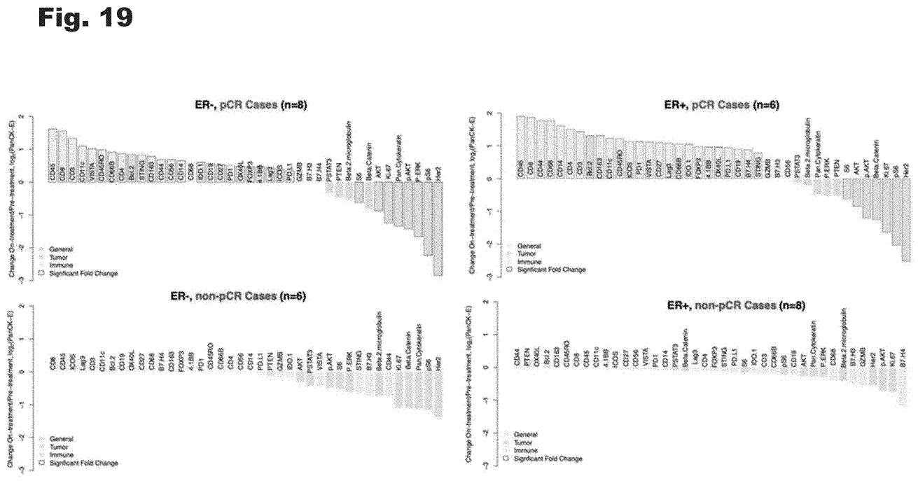

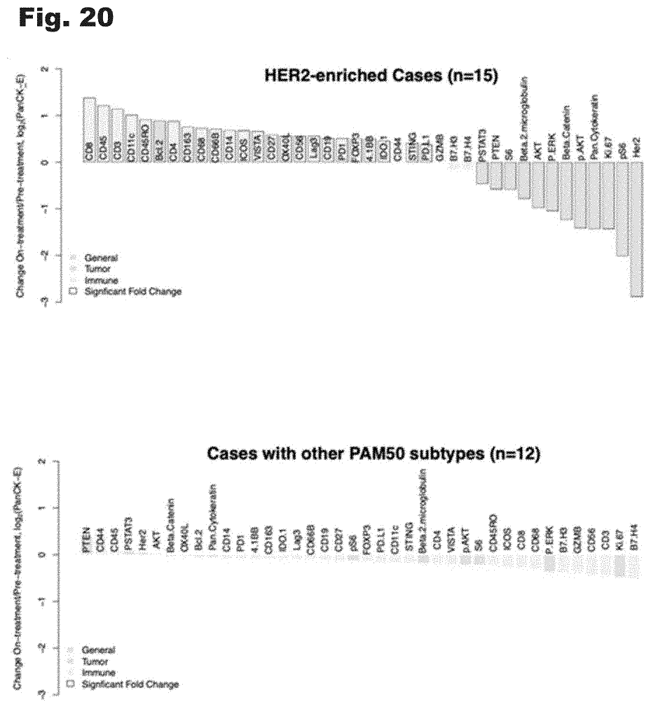

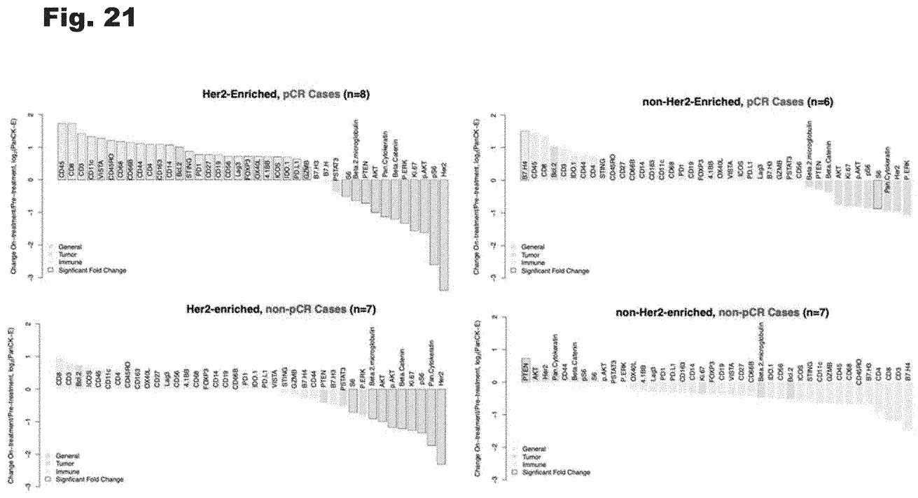

Since both ER status and HER2-enriched subtype have been associated with response to neoadjuvant therapy, the protein marker expression change with these covariates was analyzed. ER-negative tumors exhibited more significant changes on-treatment (relative to pre-treatment) compared to ER-positive tumors (mean absolute fold change ER-negative cases: 0.59, mean ER-positive cases: 0.36, two-sided Wilcoxon rank sum test p=0.0045, FIG. 18). However, when tumors were stratified by outcome, pCR cases exhibited more significant changes than non-pCR cases regardless of ER status (FIG. 19) and ER status was not predictive of pCR in this cohort (p=0.47). Similarly, tumors classified as HER2-enriched prior to treatment exhibited significant changes in tumor and immune markers in the on-treatment biopsy relative to other subtypes (FIGS. 20 and 21). For example, while CD8+ T-cells increased significantly with treatment in HER2-enriched cases, they decreased slightly in other cases. As in the full TRIO-US B07 transcriptomic cohort, HER2-enriched subtype was not predictive of pCR (p=0.87).

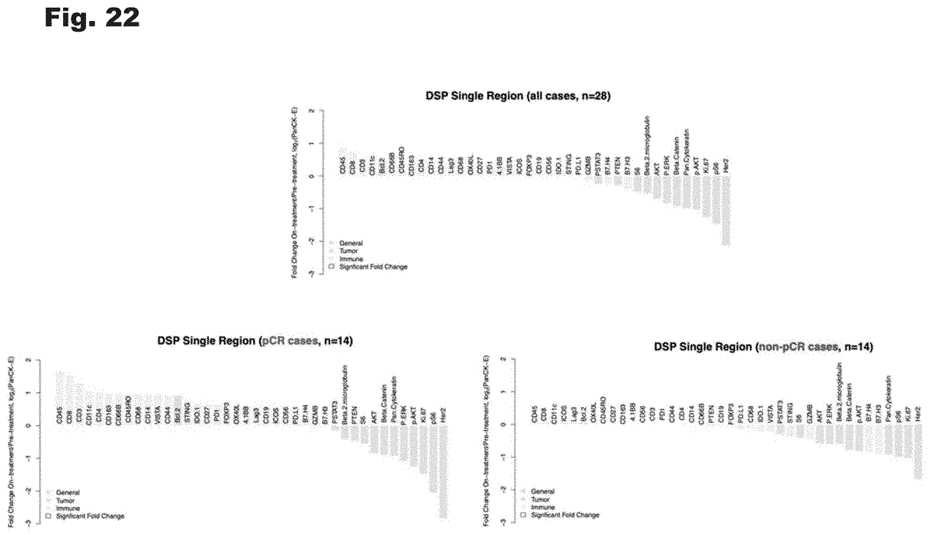

In order to assess the utility of multi-region sampling, changes on- versus pre-treatment were measured using a single randomly selected region per tissue sample averaged across 100 simulations (FIG. 22). Consistent with the findings based on all tumor regions, CD45 and CD8 showed the greatest increase on-treatment, while HER2 and pS6 decreased most in the single region analysis. While the magnitude of marker fold change with treatment was greater for pCR cases than non-pCR cases (mean absolute fold change across all markers in pCR cases: 0.87 versus non-pCR cases: 0.33, two-sided Wilcoxon rank sum test p=1.02e-07), individual markers did not increase significantly with treatment in the single region analysis, reflecting increased variance.

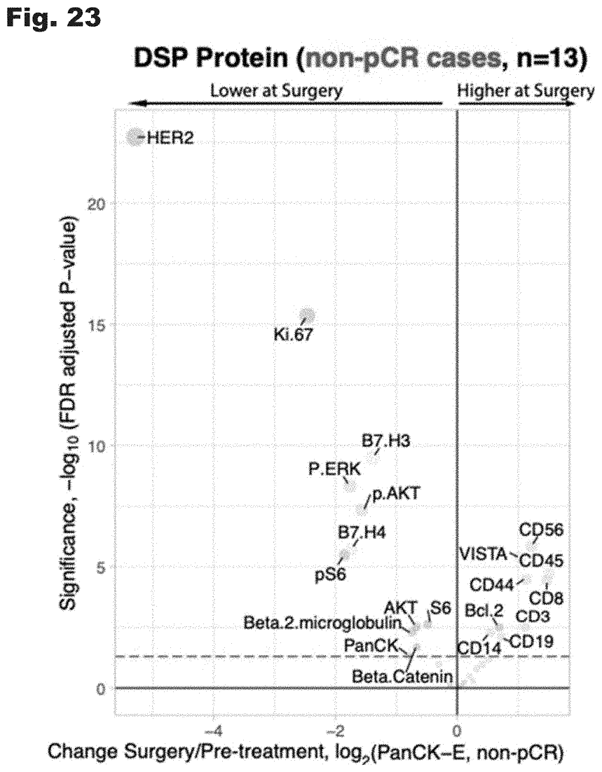

Treatment-associated changes was also examined in patients with residual tumor cells present at the time of surgery (non-pCR cases) to elucidate the biology associated with combined HER2-targeted therapy and chemotherapy. While the non-pCR cases showed limited changes at the on-treatment timepoint, by the time of surgery there was a substantial decrease in the HER2 and downstream AKT signaling pathway, and a concomitant increase in immune markers in panCK-enriched regions (FIG. 23). Notably, HER2 decreased more significantly than its downstream pathway members, which may reflect compensatory pathway activation contributing to resistance. While some immune markers increased significantly in non-pCR cases at surgery (n=8), the fold change was diminished relative to pCR cases sampled on treatment (mean fold change non-pCR post-treatment: 0.30, mean fold change pCR on-treatment: 0.85, two-sided Wilcoxon rank sum test p=0.0021, FIGS. 16A and 16B). Amongst the immune markers that increased at surgery in the non-pCR cases, CD56 was most significant and potentially related to the role of NK cells in identifying and killing chemotherapy-stressed tumor cells. NK-cells were similarly found to increase at time of surgery in the TRIO-US B07 bulk expression data.

### Increased Hheterogeneity of Tumor and Immune Markers During HER2-Targeted Therapy

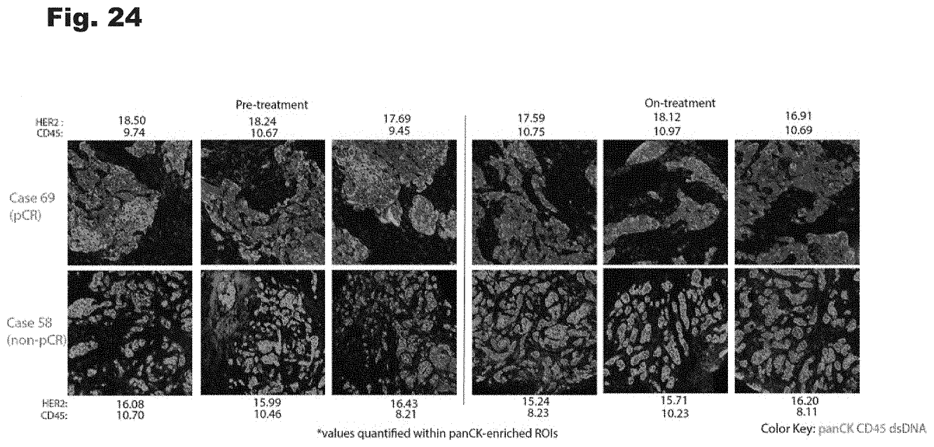

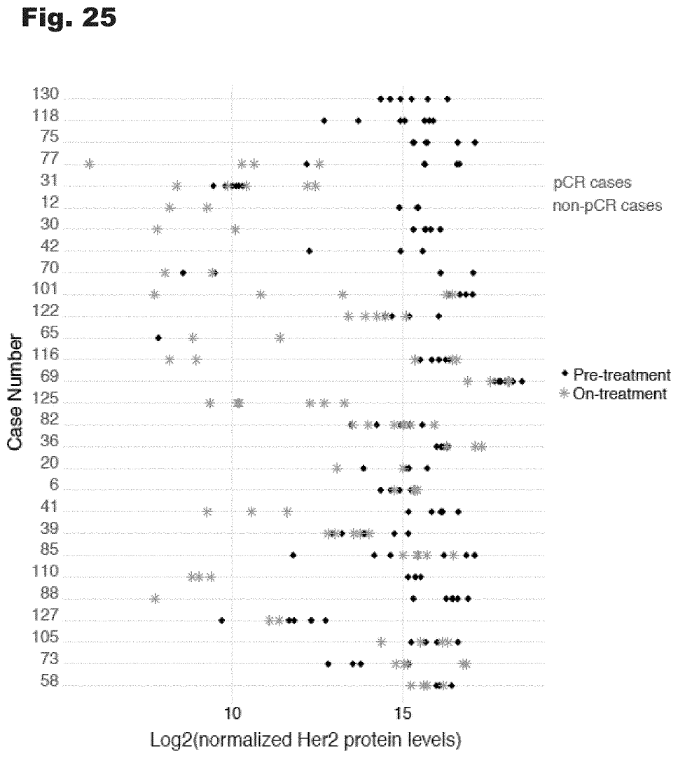

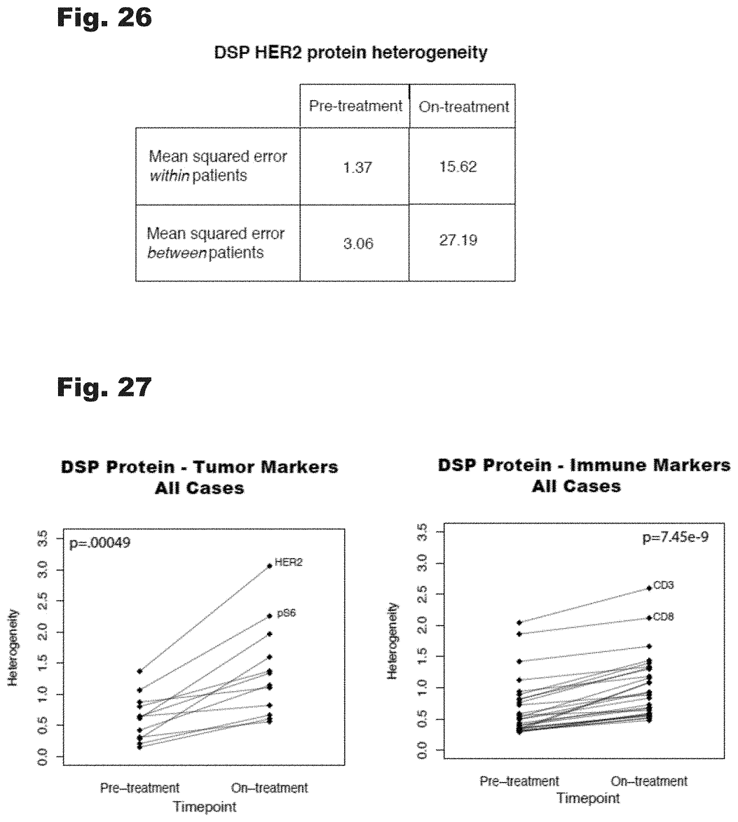

Given that tumor heterogeneity is a defining feature of HER2-positive breast cancer, the variation of HER2 protein expression within different regions of a breast tumor biopsy was examined through neoadjuvant treatment and between patients. As shown for two exemplary cases (FIG. 24), HER2 protein levels across geographically disparate regions within each tissue sample exhibited relatively consistent HER2 protein levels prior to treatment in the majority of cases (FIG. 25). Far greater heterogeneity in HER2 protein expression was observed on treatment both between regions and between patients (FIG. 26). Such regional heterogeneity may reflect pharmacokinetic differences due to vasculature, tissue architecture, immune infiltration, or the biopsy itself, underscoring the importance of profiling multiple regions per sample on-treatment.

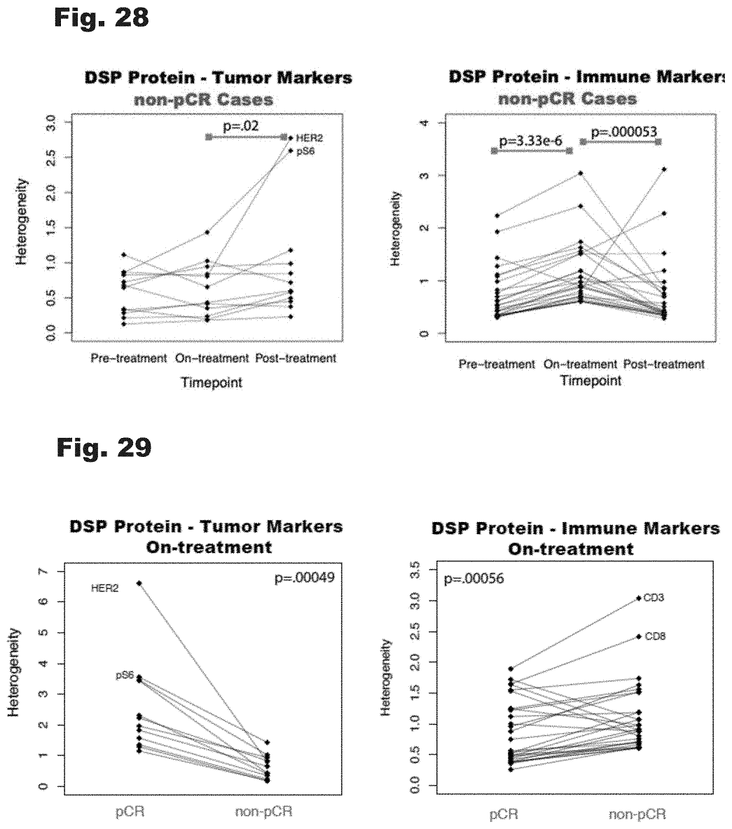

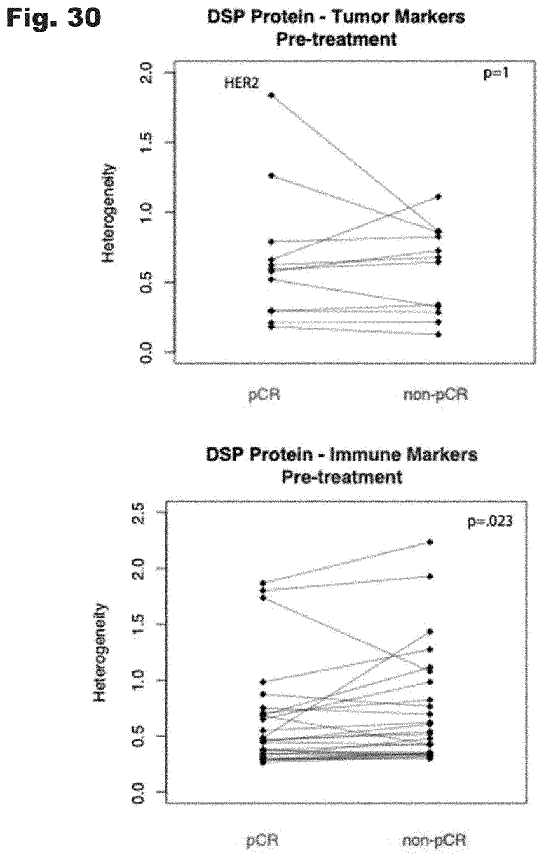

Regional heterogeneity across both tumor and immune protein markers during treatment was also investigated. For each marker and at each timepoint, regional heterogeneity across the cohort was computed as the within-patient mean squared error based on ANOVA (Methods). Across all markers, DSP protein heterogeneity increased significantly on-treatment relative to pre-treatment (FIG. 27), similar to that noted for HER2. These changes were widespread, with heterogeneity being higher for all tumor and immune markers on-treatment compared to pre-treatment. The probes with the greatest heterogeneity included both tumor (HER2, pS6) and immune (CD3, CD8) markers. Amongst tumors that failed to achieve a pCR, we evaluated heterogeneity throughout the course of neoadjuvant therapy. Heterogeneity amongst tumor markers was not significantly different on-treatment and pre-treatment (two-sided Wilcoxon rank sum test p=0.52), but increased at surgery (post-treatment), whereas immune marker heterogeneity increased on treatment with a subsequent decrease at surgery (FIG. 28). Tumors that achieved a pCR exhibited higher protein heterogeneity amongst tumor markers (including HER2) on-treatment, whereas those that did not exhibited higher heterogeneity across immune markers (FIG. 29). Higher immune marker heterogeneity on-treatment in the non-pCR cases may reflect a less consistent immune response with some regions experiencing a greater immune influx than others. Higher pre-treatment HER2 heterogeneity was not observed in the non-pCR cases compared to pCR cases. And comparable regional heterogeneity amongst tumor markers was noted in pCR and non-pCR cases (FIG. 30).

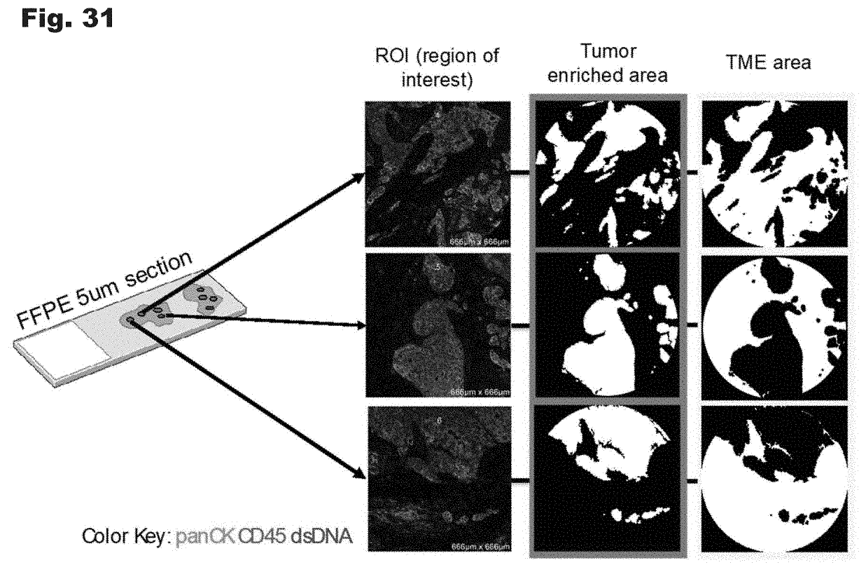

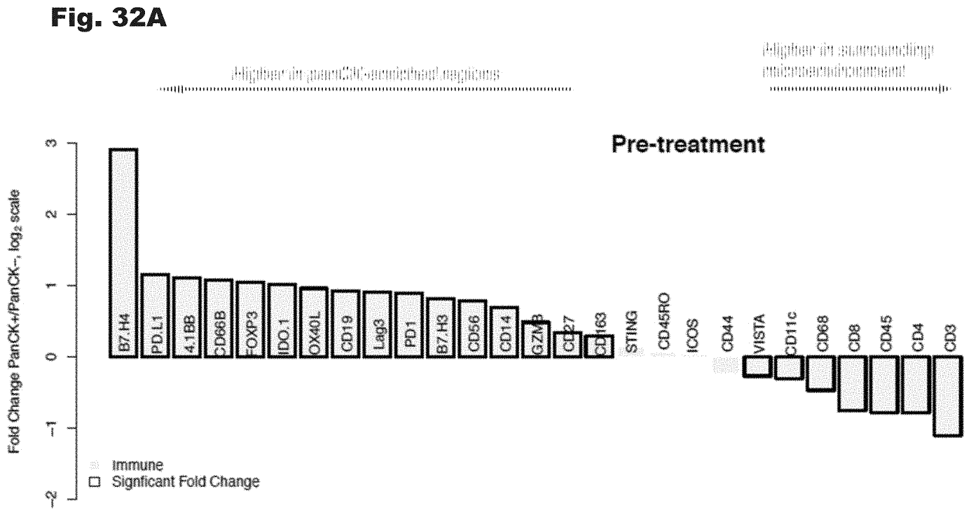

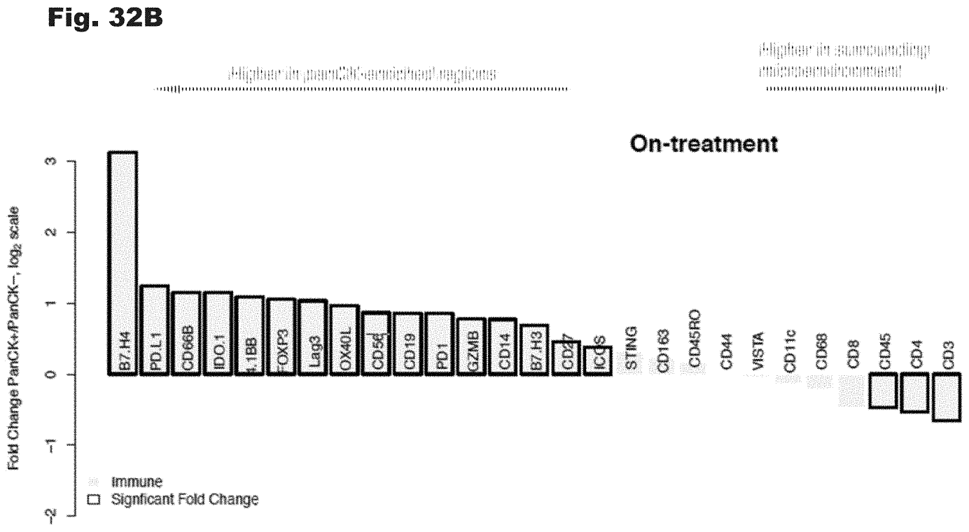

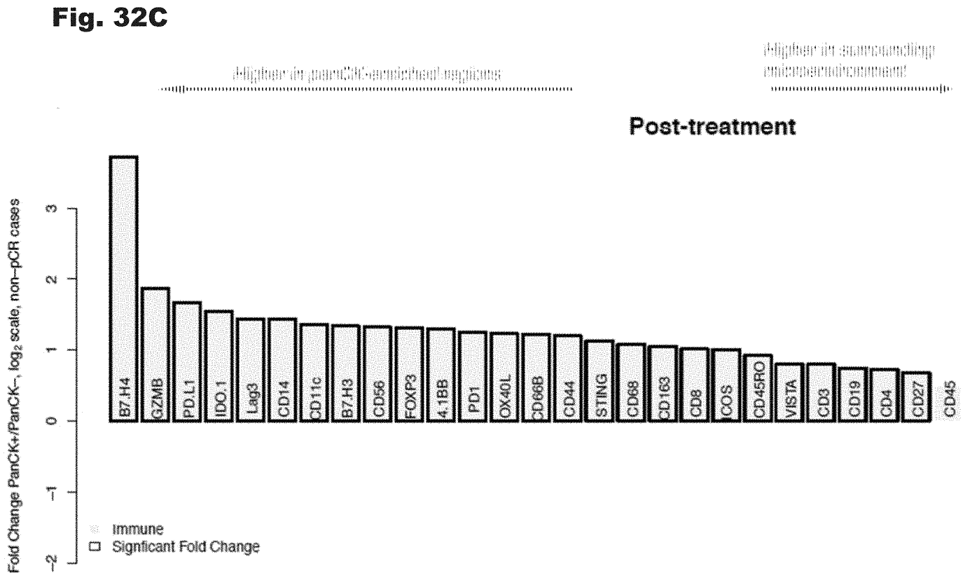

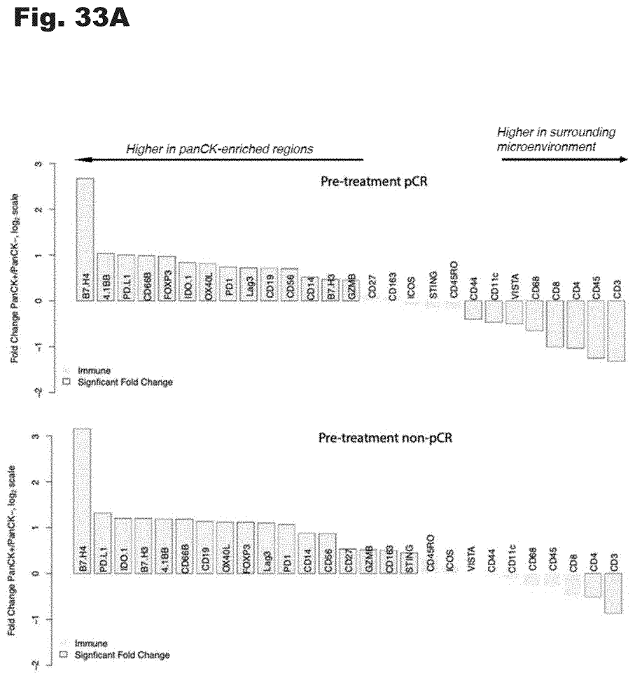

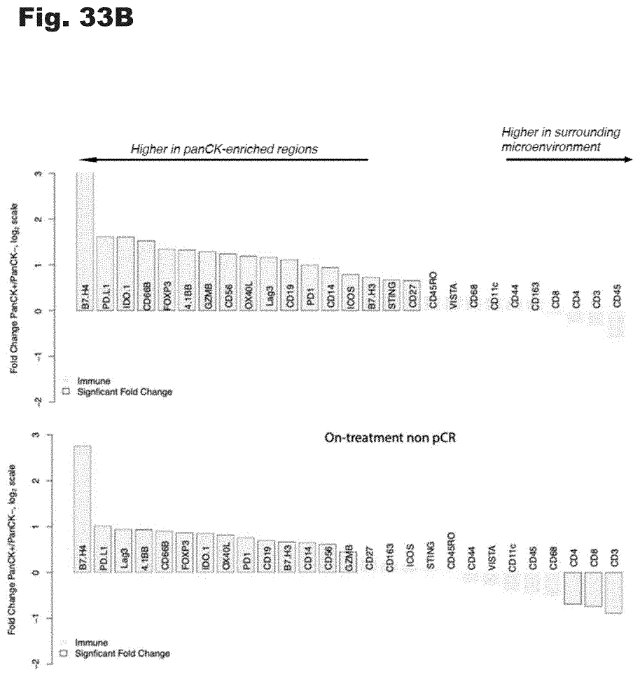

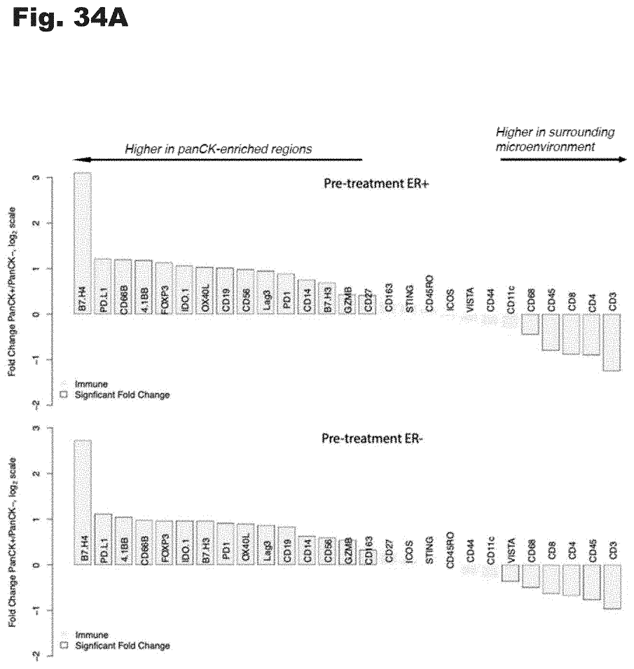

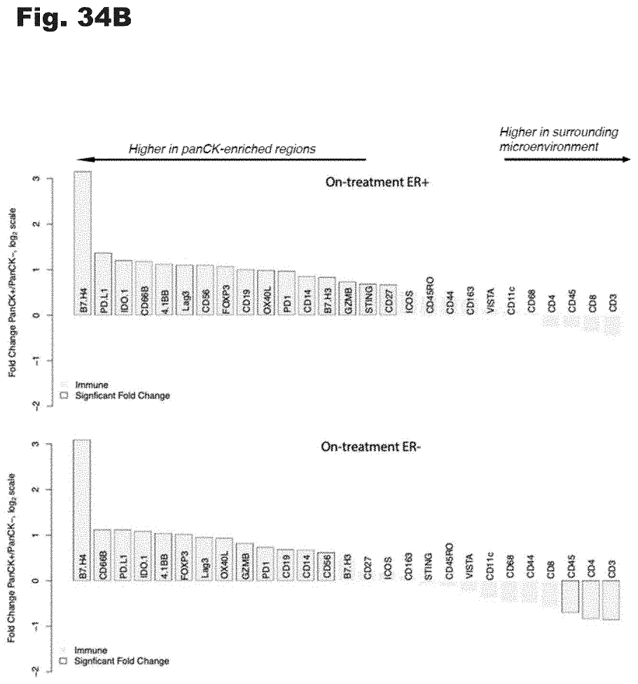

The DSP data was further analyzed to investigate the composition of immune cells in panCK-enriched regions (as used in other analyses) relative to the surrounding panCK-negative regions designed to capture the neighboring microenvironment (FIG. 31). Prior to treatment, both T cell (CD3, CD4, CD8) and macrophage (CD68) markers were more prevalent in the surrounding microenvironment, while CD56-positive NK cells and immunosuppressive markers (e.g. VCTN1, PD-L1, IDO) were higher in panCK-enriched regions (FIGS. 32A to 32C). These findings are consistent with T cell exclusion, where IDO and PD-L1 are thought to impair intratumor proliferation of effector T cells. A similar immune profile was observed during HER2-targeted therapy alone. However, post-treatment, in the non-pCR cases with tumor remaining, most immune markers were more prevalent in the panCK-enriched regions, compared to the neighboring microenvironment, including CD8 and CD68. Both prior to treatment and on-treatment, immune cell localization was similar in patients that achieved a pCR and those that did not (FIGS. 33A & 33B), as well as for ER-positive versus ER-negative cases (FIGS. 34A & 34B).

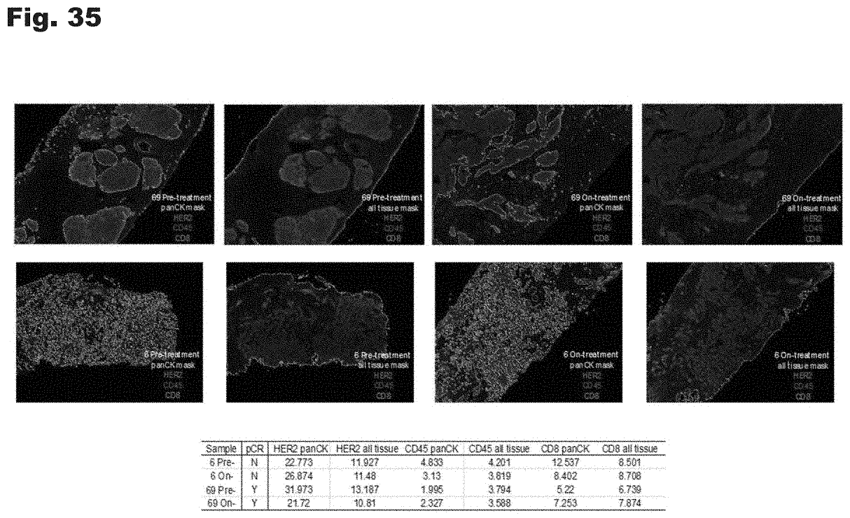

As preliminary proof of principle, noting that other multiplexed imaging technologies can similarly be used to profile panCK-enriched tumor, multiplex immunohistochemistry (mIHC) with panCK enrichment was also used to profile tissue samples from a patient that achieved a pCR and one that did not panCK antibodies were used to define mask regions and several markers that changed significantly with treatment based on DSP, namely HER2, CD45, and CD8 were quantified across the whole tissue section and within panCK-enriched regions (FIG. 35). As expected, changes in protein expression signals were muted when the whole tissue section was considered relative to panCK-enriched regions. These data further support the concept that panCK enrichment may be beneficial for defining tumor and co-localized immune changes in breast and other tumors.

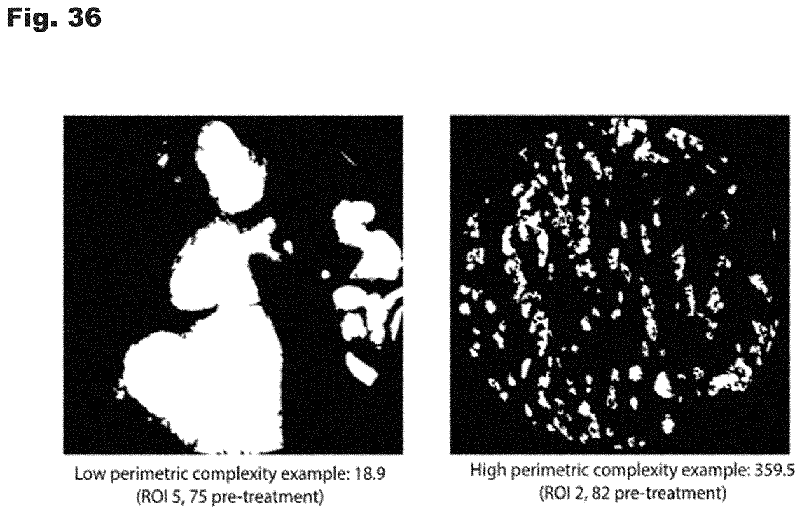

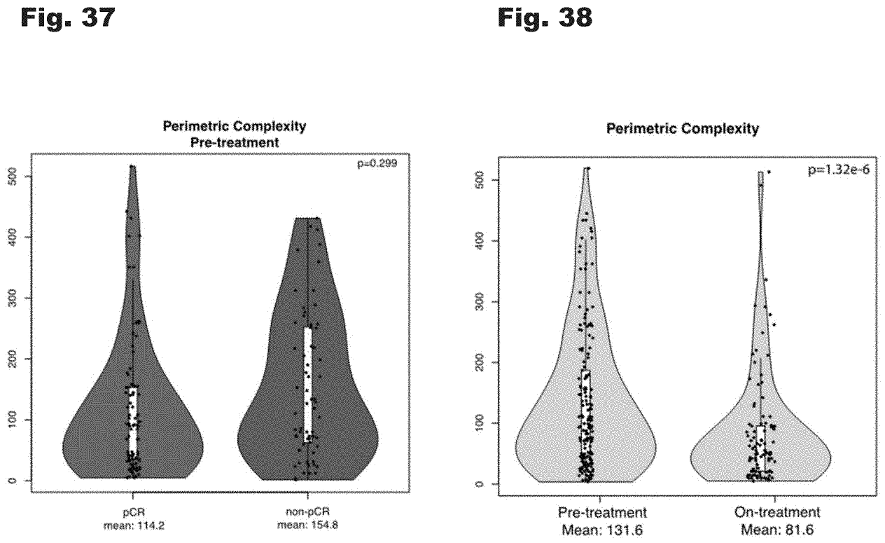

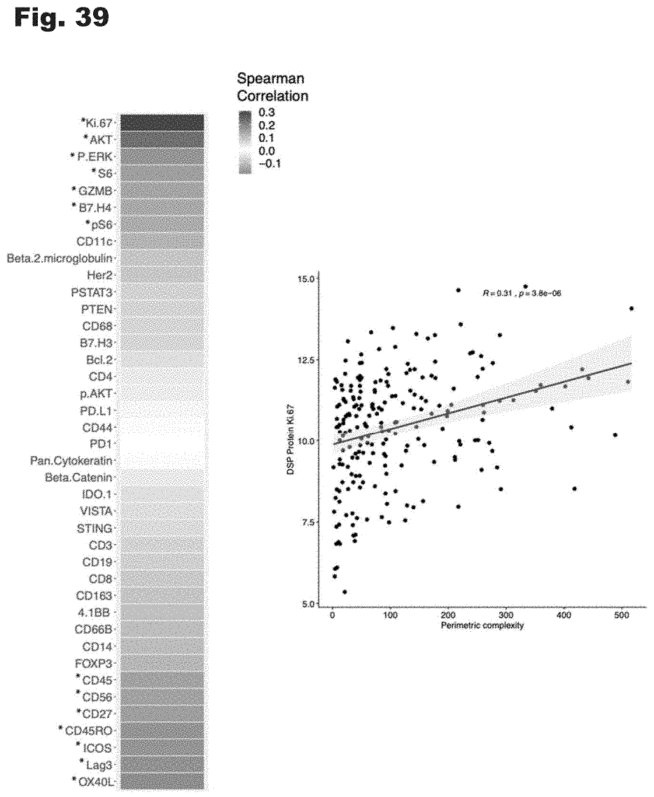

The geospatial distribution of tumor and immune cells has been associated with relapse and survival in multiple tumor types. Here, the relationship between treatment and the tumor-microenvironment border was investigated using perimetric complexity, which is proportional to the perimeter of a region squared, divided by the area of the region (Methods, FIG. 36). Prior to treatment, no significant difference in perimetric complexity was observed between pCR and non-pCR cases (p=0.299, FIG. 37). However, perimetric complexity decreased significantly on-treatment relative to pre-treatment (p=1.32e-6, FIG. 38). These data suggest that treatment may affect the geographic distribution of tumor cells as well as tumor cell content. Indeed, the proliferative marker, Ki67, was highly correlated with perimetric complexity (FIG. 39). Thus, for highly proliferative tumors, the perimeter of the tumor-microenvironment border may be relatively larger, potentially allowing for increased crosstalk with the surrounding microenvironment.

### DSP of Paired and Pre- and On-Treatment Biopsies Reveals Features Associated with pCR

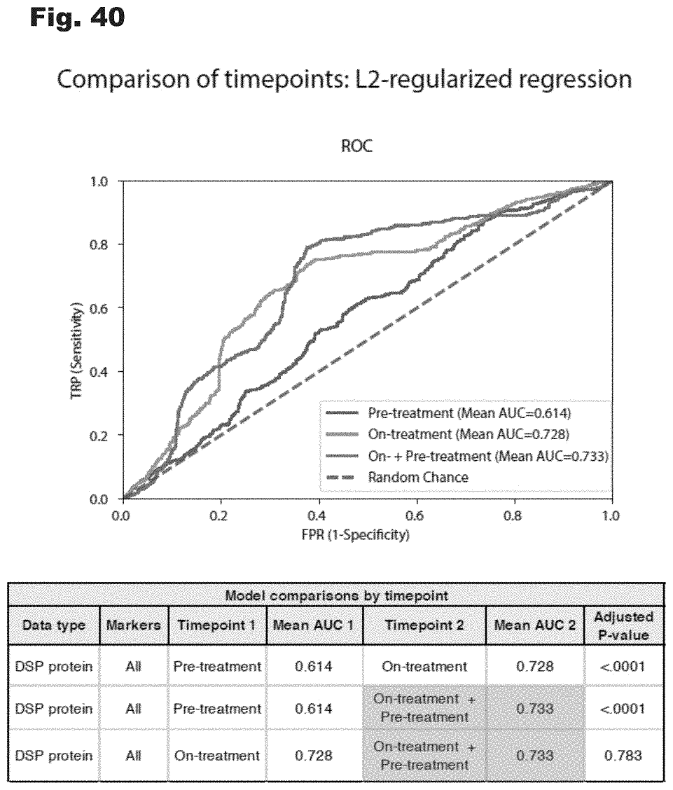

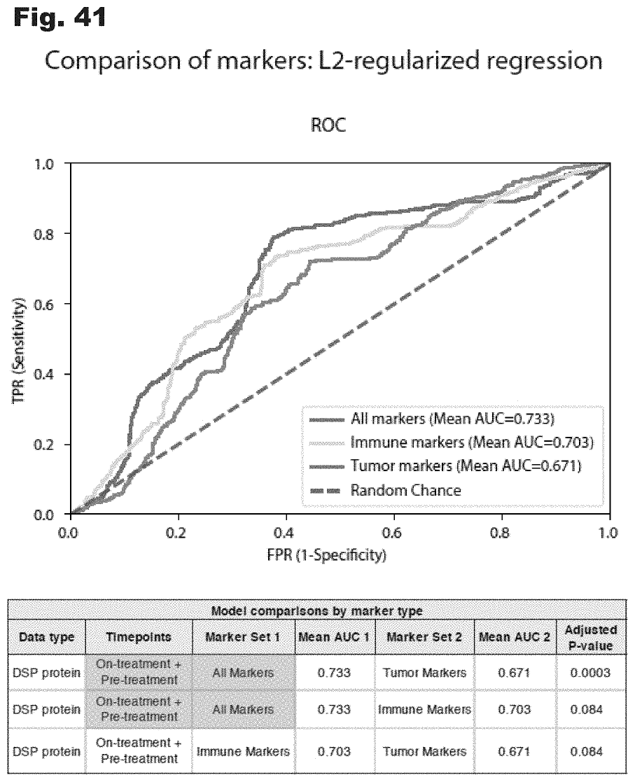

Given the dramatic differences in treatment-associated changes in pCR cases compared to non-pCR cases (FIGS. 16A & 16B), it was next sought to evaluate whether DSP protein marker status prior to treatment or early during the course of therapy could be used to predict pCR. An L2-regularized logistic regression was used to classify tumors by pCR status based on average DSP protein expression levels across multiple ROIs profiled pre-treatment, on-treatment, or the average marker expression both pre-treatment and on-treatment (denoted “on- +pre-treatment”) and evaluated model performance via nested cross validation within the discovery cohort (Methods). Tumors with data at both timepoints were utilized in this analysis (n=23 cases, FIG. 4). A model based on on-treatment protein expression outperformed that based on pre-treatment protein expression (mean AUROC=0.728 versus 0.614) and performed comparably to a model incorporating both on-treatment and pre-treatment protein expression levels (mean AUROC=0.733) (FIG. 40). A classifier trained using both immune and tumor markers outperformed a model using tumor markers alone, highlighting the utility of simultaneous tumor and immune profiling to predict therapy response (FIG. 41).

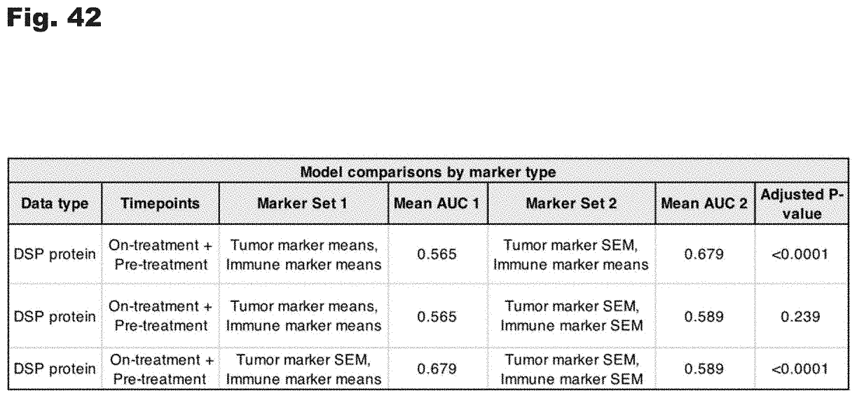

For the DSP protein on- plus pre-treatment classifier, the importance of multi-region sampling and heterogeneity was investigated by extending the model to incorporate both the mean marker expression across all regions and the standard error of the mean (SEM) for each marker between regions (Methods). This analysis was restricted to patients with at least 3 regions profiled at both timepoints (n=16, Methods). It was found that utilizing the mean immune values and the SEM for tumor markers outperformed a model based on mean values for both tumor and immune markers (FIG. 42), suggesting that classifiers that capture the heterogeneity amongst tumor markers may improve the prediction of pCR.

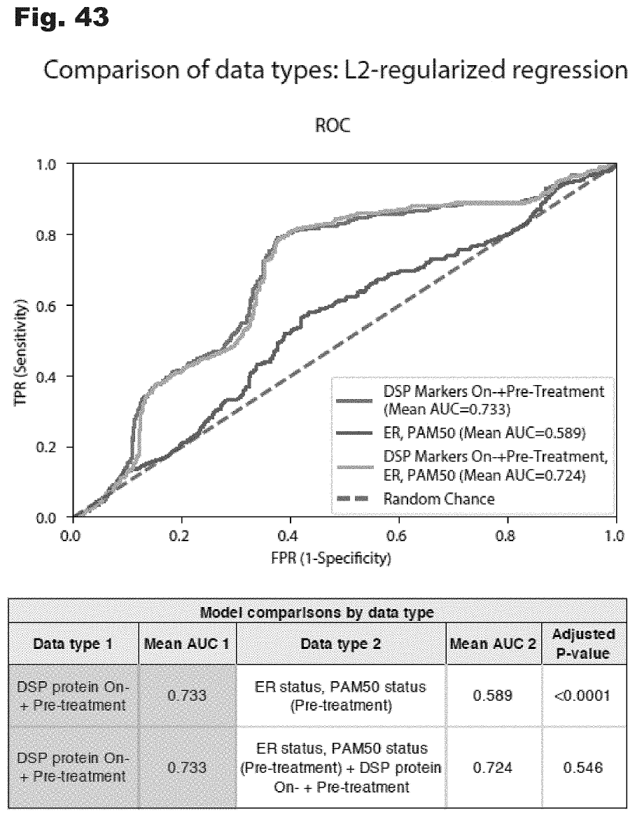

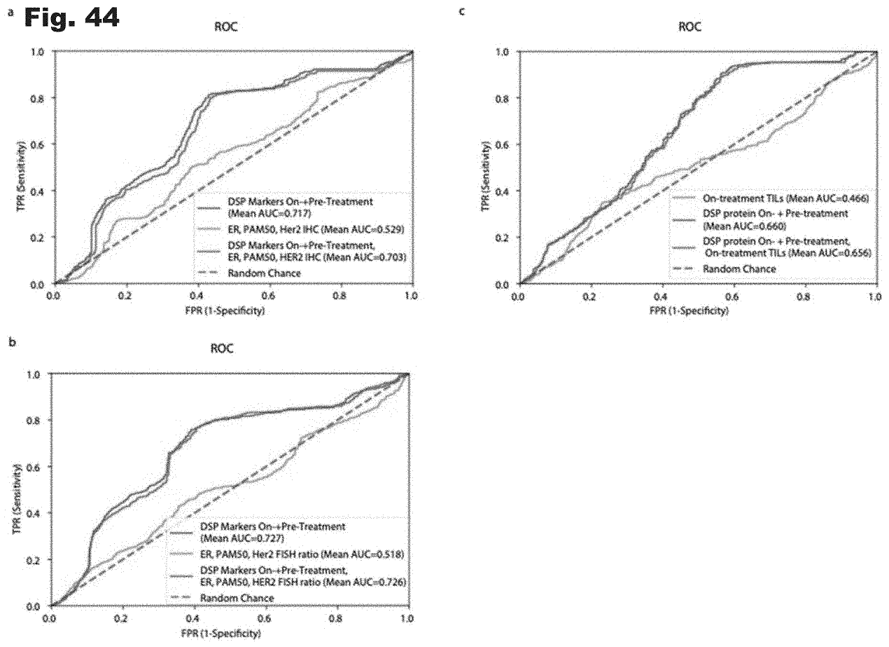

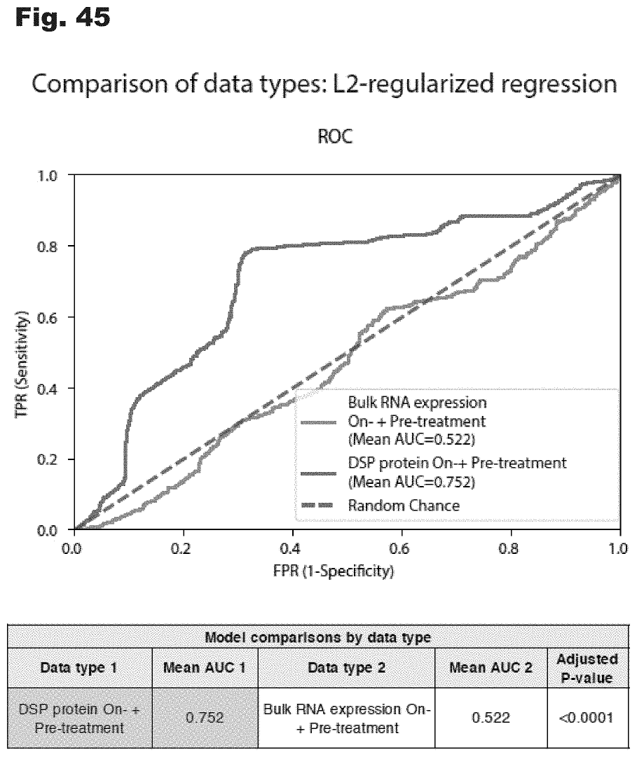

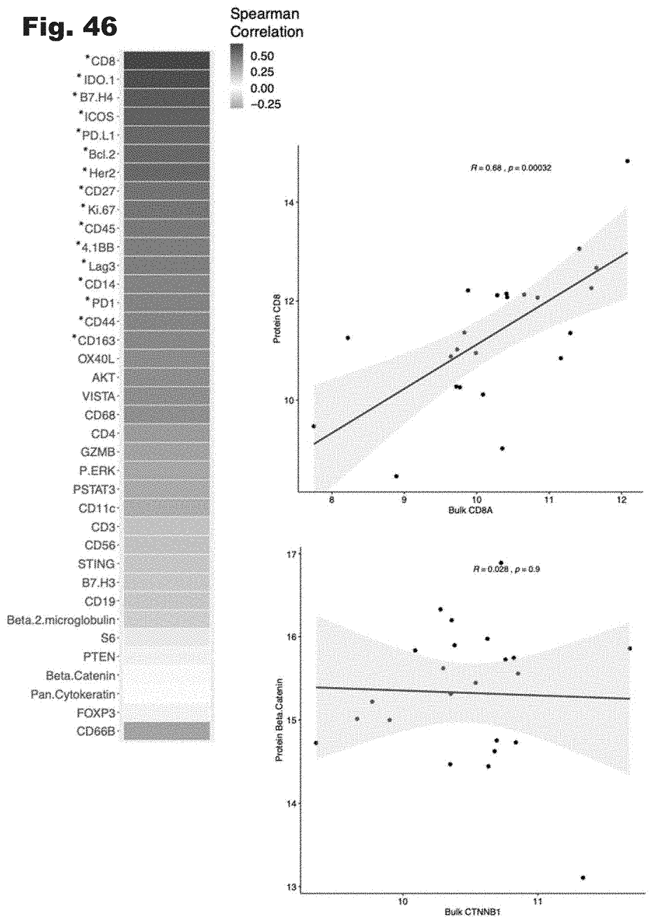

The performance of the DSP protein on- plus pre-treatment classifier was compared with features previously associated with outcome (ER status and PAM50 subtype), where models were again evaluated via cross-validation in the discovery cohort. Of note, a model based on ER status and HER2-enriched PAM50 status performed poorly in this cohort (mean AUROC=0.589) and the addition of these two features or additional pathologic features to the DSP protein on- plus pre-treatment data set did not improve the AUROC (FIGS. 43 & 44). Given the availability of bulk transcriptomic data for these cases, a model was also built using paired on- and pre-treatment bulk RNA expression data for the 37 markers that overlapped with the DSP protein panel. This model also performed significantly worse than that based on the DSP protein data (FIG. 45, p<0.0001 via cross-validation). This is not surprising since amongst the 37 overlapping DSP and bulk RNA expression markers, only 16 were positively correlated pre-treatment (FIG. 46). Various factors may contribute to the lack of strong correlation between protein and RNA expression levels, including panCK enrichment, RNA transience/degradation, and post-translational regulation, where protein expression is a more proximal readout of cellular phenotype.

### DSP Predicts pCR in an Independent Validation Cohort

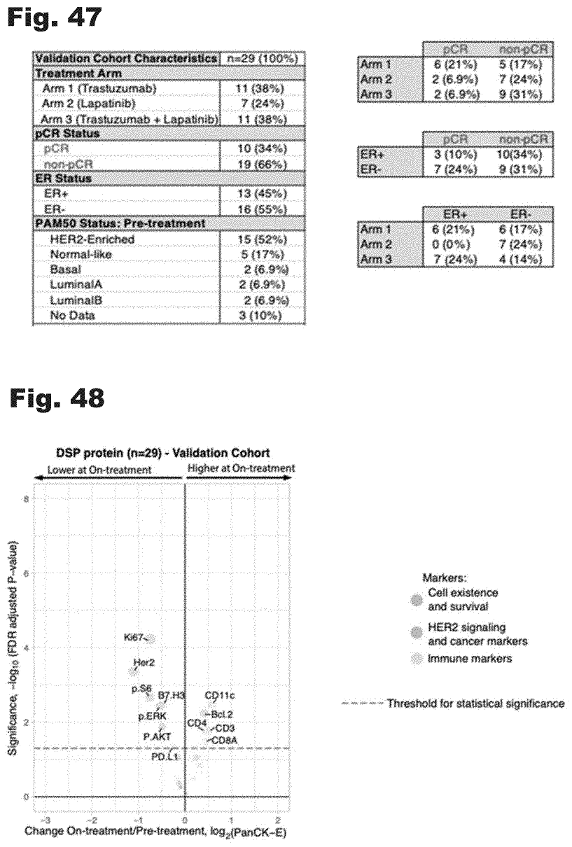

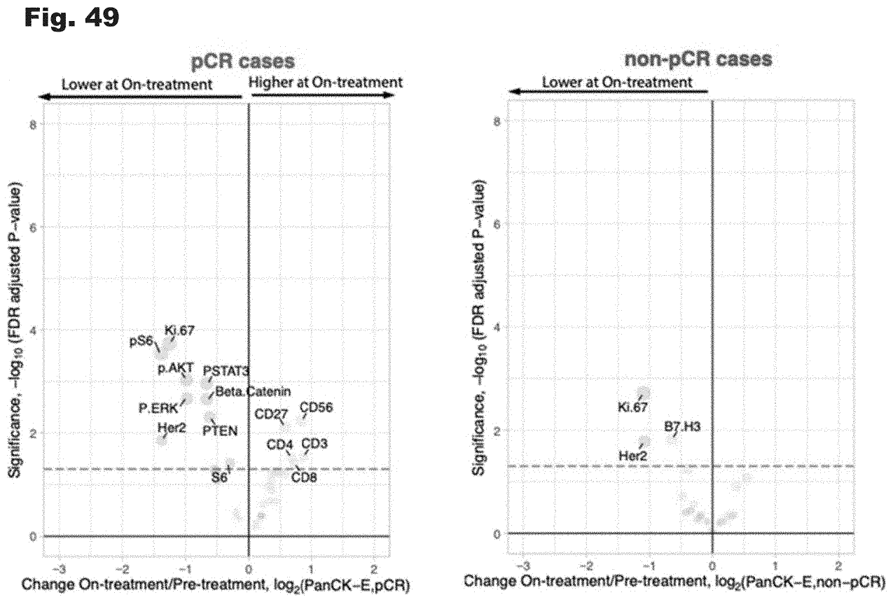

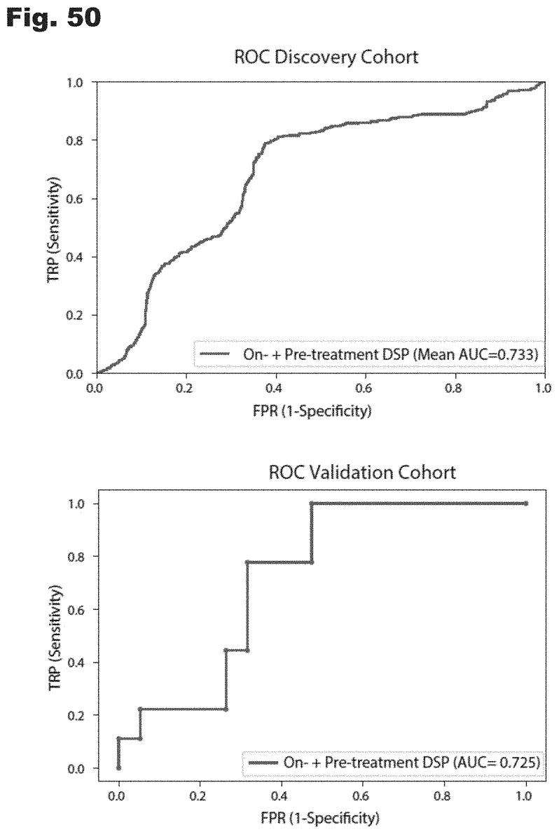

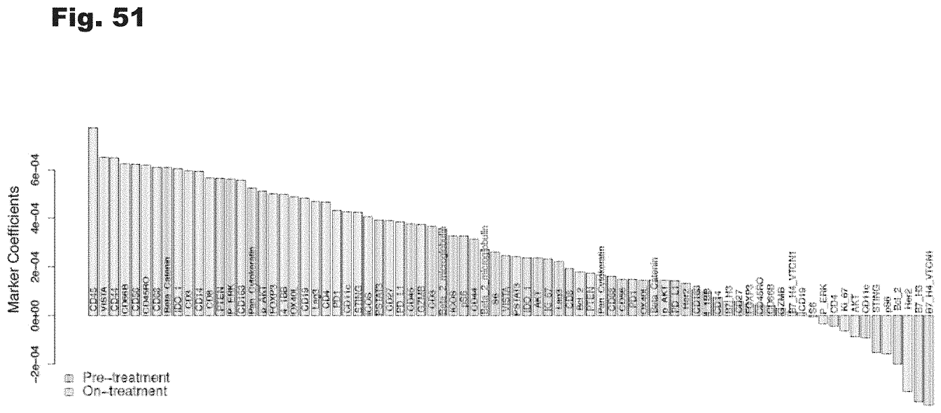

In light of these promising findings, it was further sought to evaluate the performance of the DSP protein on- plus pre-treatment classifier in an independent cohort (n=29) of patients from the TRIO B07 clinical trial (FIG. 47). As with the discovery cohort, an average of four panCK-positive regions were profiled from each pre- and on-treatment tumor tissue and the same panel of 40 protein antibodies was utilized. The change in markers on-treatment relative to pre-treatment mirrored that observed in the discovery cohort (FIG. 48): T-cell markers (CD3, CD4, and CD8) increased while the tumor markers HER2 and Ki67 showed the most significant decrease. Similar to the discovery cohort, in the validation cohort, the differences between on-treatment and pre-treatment protein expression were more dramatic in tumors that ultimately underwent pCR (FIG. 49). AUROC performance of the L2-regularized logistic regression model, trained in the discovery cohort, was evaluated in the validation (test) cohort. The performance of the DSP protein on- plus pre-treatment model in predicting pCR was comparably high in the discovery (assessed via cross-validation, mean AUROC=0.733) and validation (assessed via train-test, AUROC=0.725) cohorts (FIG. 50). In the on- plus pre-treatment classifier, which was trained in the discovery cohort and tested in the validation cohort, the marker with the largest L2-regularized coefficient was on-treatment CD45 protein levels. In general, features with large coefficients included on-treatment markers that represent tumor-infiltrating lymphocyte and macrophage populations (CD45, CD44, CD66B) (FIG. 51). On-treatment HER2 protein expression had a negative coefficient in the model, consistent with poor outcome being associated with high HER2 levels during treatment.

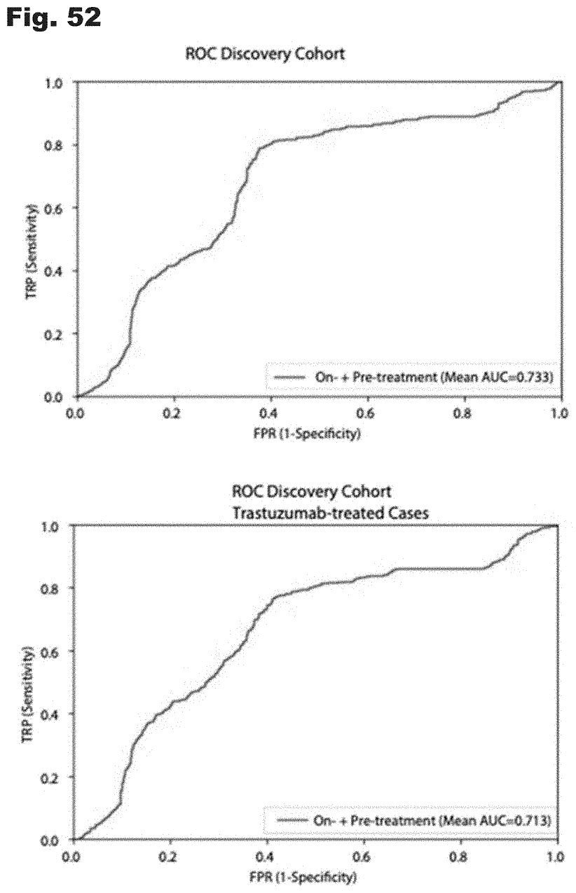

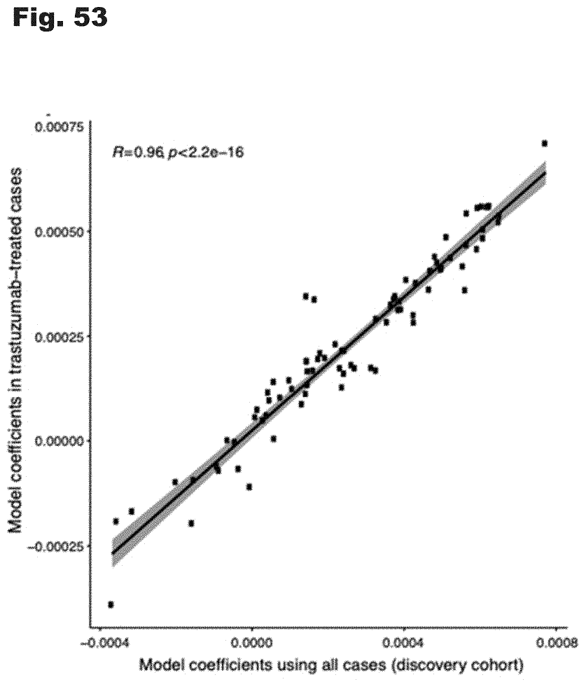

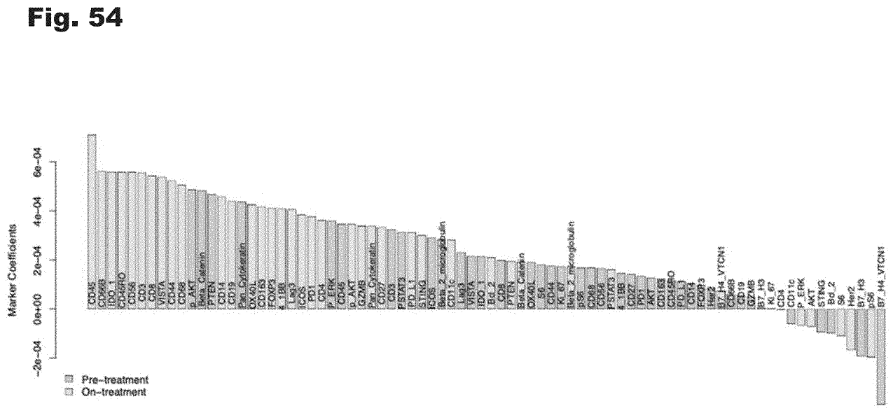

Given the widespread use of trastuzumab in current neoadjuvant treatment paradigms, model performance was further assessed for the best performing on- +pre-treatment DSP protein model in the trastuzumab-containing cases (arms 1 and 3, n=19). Similar model performance and marker coefficients was observed in the full discovery cohort and in the subset of cases in the validation cohort who were treated with trastuzumab (FIGS. 52 to 54). The validation of these findings in an independent cohort demonstrates the potential of multiplex spatial proteomic profiling to predict which patients will respond early during HER2-targeted therapy, such that subsequent therapy could be tailored accordingly.

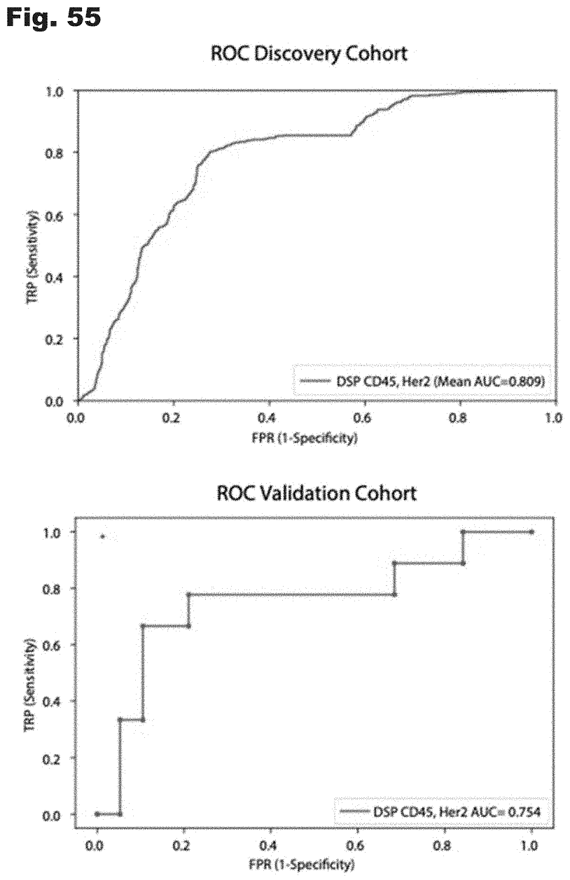

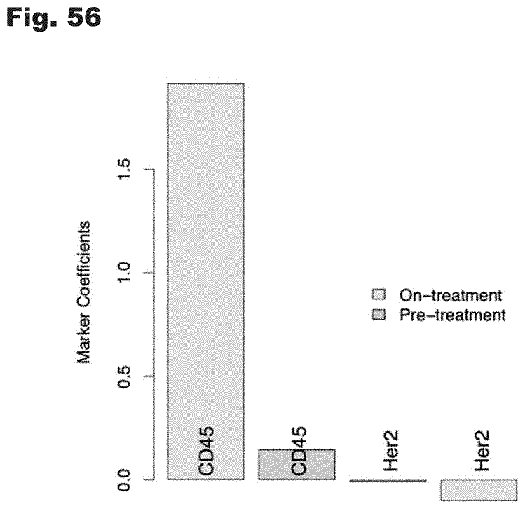

Two markers of biological significance, CD45 and Her2, were selected to assess model performance using a reduced marker set. Again, an L2-regularized logistic regression model was trained in the discovery cohort, and was evaluated in the validation (test) cohort. The performance of the DSP HER2, CD45 on- plus pre-treatment model in predicting pCR was high in both the discovery (assessed via cross-validation, mean AUROC=0.809) and validation (assessed via train-test, AUROC=0.754) cohorts (FIG. 55). As with the full marker panel, CD45 on-treatment had the greatest coefficient (FIG. 56).

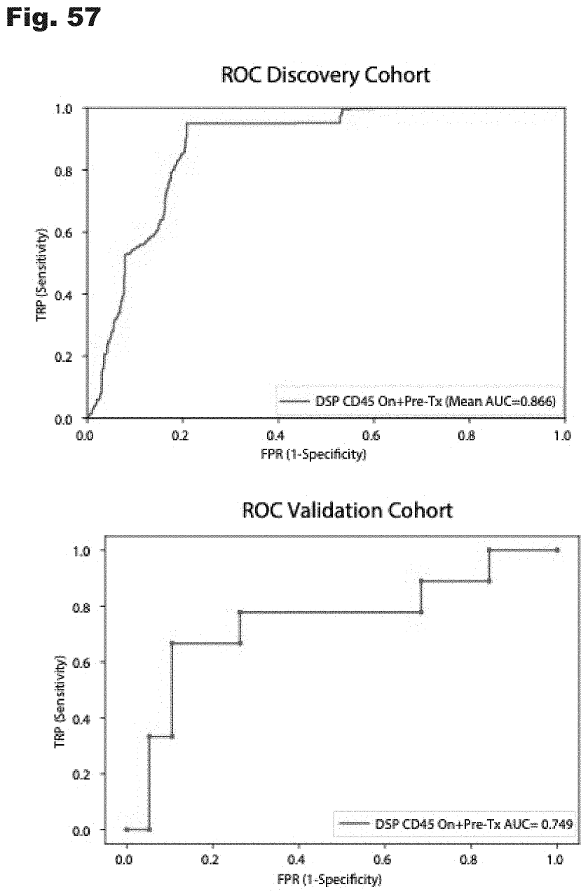

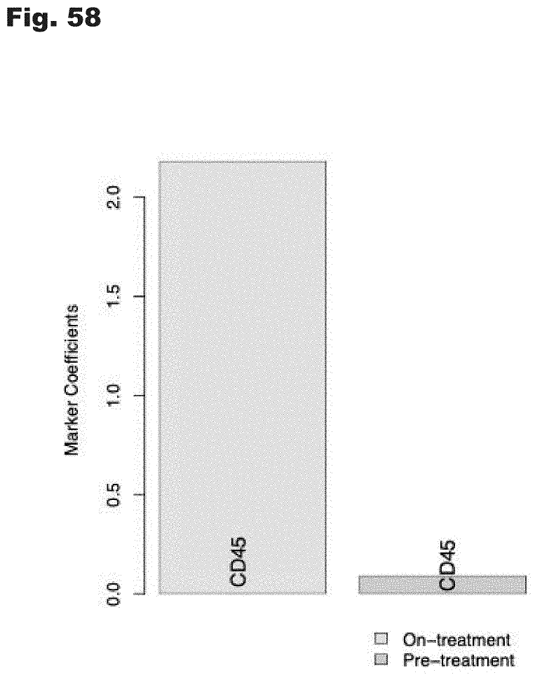

Finally, a model was built based upon solely CD45 and the performance was assessed. An L2-regularized was trained in the discovery cohort, and was evaluated in the validation (test) cohort. The performance of the CD45 on- plus pre-treatment model in predicting pCR was high in both the discovery (assessed via cross-validation, mean AUROC=0.866) and validation (assessed via train-test, AUROC=0.749) cohorts (FIG. 57). Again, CD45 on-treatment had the greatest coefficient (FIG. 58).

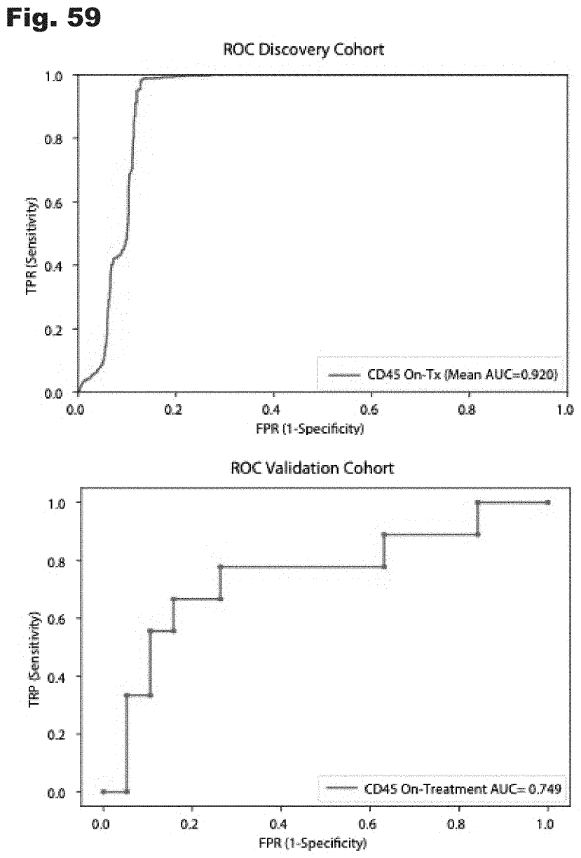

An L1-regularized model based on CD45 was also trained on the discovery cohort, and was evaluated in the validation (test) cohort. The performance of the CD45 on-treatment model in predicting pCR was high in both the discovery (assessed via cross-validation, mean AUROC=0.920) and validation (assessed via train-test, AUROC=0.749) cohorts (FIG. 59).

### DISCUSSION

Bulk genomic and transcriptomic profiling has been a mainstay of cancer biomarker discovery efforts in recent years. However, admixture amongst heterogeneous cellular populations complicates the analysis of such data, issues which are compounded when studying longitudinal samples, where the changing composition and localization of cell populations may reflect the biology of disease progression or mechanisms of treatment response. Indeed, efforts to establish validated biomarkers of response to HER2-targeted therapy based on bulk genomic and transcriptomic profiling have met with limited success to date in other trial cohorts and in TRIO-US B07. It was reasoned that in situ proteomic profiling of the tumor-immune microenvironment through therapy would circumvent the limitations of dissociative techniques and improve the ability to uncover features associated with response to neoadjuvant HER2-targeted therapy. Here, the DSP technology was used to simultaneously profile 40 tumor and immune markers en bloc on a single 5 μm section of archival tissue from breast tumors sampled before, during, and after neoadjuvant HER2-targeted therapy in the TRIO-US B07 clinical trial. In order to enhance signal while accounting for intra-tumor heterogeneity, a pan-CK masking strategy was employed to enrich for tumor cells and co-localized immune cells across multiple regions per sample.

DSP of longitudinal breast biopsies from this trial cohort uncovered changes associated with therapy, including markedly decreased HER2 and downstream AKT signaling on-treatment, accompanied by increased CD45 and CD8 expression, consistent with infiltrating leukocytes and cytotoxic T-cells, respectively. By the time of surgery, following a full course of neoadjuvant therapy, the tumor-immune composition changed considerably with increased CD56 expression in non-pCR cases, potentially reflecting NK cell-mediated killing of chemotherapy-stressed tumor cells. Changes in both tumor and immune markers on-treatment were more dramatic in tumors that went on to achieve a pCR and, critically, on-treatment and pre-treatment protein expression robustly predicted response in an independent validation cohort (AUROC=0.725). Whereas on-treatment protein expression levels were similarly predictive of pCR, highlighting that in future studies profiling of on-treatment tissue alone may be sufficient to predict subsequent pCR, neither pre-treatment protein expression, established predictive features, nor bulk pre-and on-treatment gene expression data were predictive in this cohort, emphasizing the superiority of this novel multiplexed spatial proteomic biomarker and its potential utility for patient stratification. These findings thus address a critical unmet clinical need given the considerable emphasis devoted to identifying subsets of the population in which therapy should be escalated, for example by combining HER2-targeted agents, or safely de-escalated, for example through shortening or omission of chemotherapy and its associated toxicities. While numerous biomarkers have been considered to help guide personalized targeting of escalated versus de-escalated approaches in early-stage HER2+ breast cancer—including imaging, circulating tumor DNA, and pre-treatment immune scores or intrinsic subtype—there is currently no validated biomarker that can guide patient stratification. The increasing plethora of options for HER2-targeted therapy, including novel highly effective but potentially toxic agents, combined with great heterogeneity in response make HER2+ breast cancer the ideal setting for the development of optimally personalized therapy over the next decade.

More generally, the results illustrate the feasibility and power of multiplex in situ proteomic analysis of archival tissue samples to provide proximal readouts of tumor and immune cell signaling through therapy. Many signaling proteins/phospho-proteins, including those profiled here, are considered protein network bottlenecks and integrate mutational and transcriptional changes, making this a particularly powerful approach to studying treatment-associated changes. Importantly, DSP antibody panels can now be customized, allowing for inclusion of additional/alternate markers of interest, such as ER or other tumor-specific markers and signaling pathways. This work also illuminates study design considerations, including the value of panCK enrichment of tumor cells (or other markers to enrich for specific cell populations) and multi-region profiling to capture regional tumor heterogeneity and treatment-associated changes, concepts that should be broadly applicable to other epithelial tumor types. This together with the quantitative and multiplex nature of DSP represents a notable difference compared to classical IHC. Of note, the DSP measurements in this study were based on regional analysis of defined cellular populations comprised of ˜300-600 cells. Although single cell resolution was not necessary for the development of the novel biomarker described within (and may indeed complicate the clinical implementation of such approaches), such data can further enable the identification of cell states and cell-cell interactions and is likely to be an area of future research, fueled by advances in the resolution and throughput of spatial profiling technologies.

### METHODS

### Cohort Selection

The TRIO-US B07 clinical trial was a randomized, multicenter study that included 130 women with stage I-III unilateral, HER2-positive breast cancer (S. Hurvitz, et al., (2020), cited supra). The IRB at the University of California Los Angeles (UCLA) approved the clinical trial TRIO-US B07 (08-10-035). The IRB at Stanford approved the use of the TRIO-US B07 clinical trial specimens for correlative studies in the Curtis Lab (eProtocol #32180). Informed consent was obtained from all participants. This covers consent from patients for their samples to be shared with other researchers. Enrolled patients were randomly assigned to three treatment groups, dictating the type of targeted therapy namely trastuzumab, lapatinib, or trastuzumab and lapatinib in combination. Breast tumor biopsies were obtained prior to treatment and following 14-21 days of the assigned HER2-targeted therapy (without chemotherapy), which was followed by six cycles of the assigned HER2-targeted treatment plus docetaxel and carboplatin given every three weeks and surgery. For each timepoint, core biopsies or surgical tissue sections were obtained and stored as either fresh frozen or FFPE material. In total, 28 cases with FFPE samples available from all three timepoints (pre-treatment, on-treatment, and at surgery) were selected for inclusion in the discovery cohort based on sample availability and quality, with balancing by pCR status and ER status (FIGS. 2 & 3). An additional 29 cases with FFPE samples available pre-treatment and on-treatment were selected for the validation cohort in order to assess performance of the classifier (FIGS. 47 to 49). Of note, the validation cohort was used exclusively to evaluate model performance. All other analyses are based on the discovery cohort. Tumor cellularity was assessed by a board-certified breast pathologist (GRB) using tumor sections stained with hematoxylin and eosin. Samples with cellularity of 0 were omitted from further analysis. For other tissue sections estimated to have a cellularity of 0, tumor cells were identified on the FFPE sections used to perform DSP (distinct from the H&E sections used for pathology review), and these were included in the analysis (FIG. 4). FFPE blocks were sectioned at 5 μm thickness and stored at 4° C. for less than three weeks prior to the DSP experiment.

### DSP Data Generation and Analysis

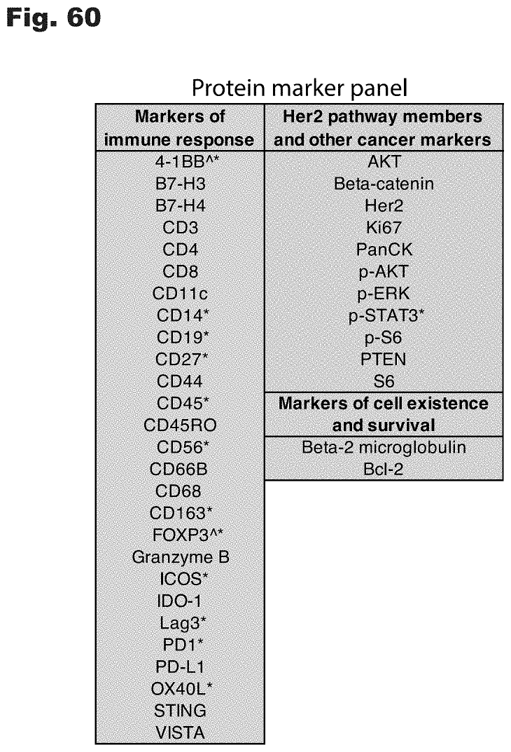

Digital Spatial Profiling (DSP, NanoString FOR RESEARCH USE ONLY. Not for use in diagnostic procedures) was performed as previously described (C. R. Merritt, et al., (2020), cited supra). In brief, tissue slides were stained with a multiplexed panel of protein antibodies contained a photocleavable indexing oligo, enabling subsequent readouts (FIG. 5). Regions of interest (ROIs) were selected on a DSP prototype instrument and illuminated using UV light. Released indexing oligos from each ROI, which were collected and deposited into designated wells on a microtiter plate, allowing for well indexing of each ROI during nCounter readout (direct protein hybridization). Custom masks were generated using an ImageJ pipeline, as described previously (R. N. Amaria, et al., Nat Med 24, 1649-1654 (2018), the disclosure of which is incorporated herein by reference). For each tissue sample, counts for each marker were obtained from an average of four (range 1-7) panCK-enriched (panCK-E) ROIs. Raw protein counts for each marker in each ROI were generated using nCounter (V. A. Malkov, et al., BMC Res Notes 2, 80 (2009), the disclosure of which is incorporated herein by reference). The raw counts were ERCC-normalized (based on the geometric mean of the three positive control markers). Histone H3 was used as a housekeeping marker and ROIs with extreme Histone H3 (more than three standard deviations away from the mean) were filtered (<1% of ROIs). The geometric mean of two IgG antibodies were used to calculate the background noise and we noted markers with signal to noise ratio <3×(FIG. 60). Immune markers were normalized based on ROI area to measure total density of immune content in the region. Tumor markers were normalized using the housekeeping antibody (Histone H3) in order to capture the status of the cancer signaling pathways on a per cell basis. As further quality control, area normalization factors and housekeeping normalization factors were compared per ROI, and ROIs were filtered with disparate normalization factors across the two methods (this represented 6% of all ROIs). All normalized counts were converted to log2 space for downstream analysis. The analyses carried out in this study are comparative in nature (e.g. pre-treatment vs on-treatment, pCR vs non-pCR) and are robust to variations in normalization methods.

### Bulk mRNA Expression Analysis

RNA was extracted using the RNeasy Mini Kit (Qiagen), quantified by the Nanodrop One Spectrophotometer (ThermoFisher Scientific). RNA samples were labeled with cyanine 5-CTP or cyanine 3-CTP (Perkin Elmer) using the Quick AMP Labeling Kit (Agilent Technologies). Gene Expression Microarray experiments were performed by comparing each baseline sample to samples taken after 14-21 days of HER2-targeted therapy (on-treatment). Each on-treatment sample was compared to the pre-treatment sample from the same patient. Limma (M. E. Ritchie, et al., Nucleic acids research 43, e47 (2015); and M. E. Ritchie, et al., Bioinformatics 23, 2700-2707 (2007); the disclosures of which are each incorporated herein by reference) was used for background correction (“normexp”), within-array normalization (“loess”), between-array normalization, and for averaging over replicate probes. For the downstream analyses, including batch correction and comparisons with the DSP cohort, the normalized counts were converted to log2 space. Combat (W. E. Johnson, et al., Biostatistics 8, 118-127 (2007), the disclosure of which is incorporated herein by reference) was used to remove potential batch affects associated with microarray run date. PAM50 status pre-treatment and on-treatment was inferred using AIMS (Absolute Intrinsic Molecular Subtyping), an N-of-1 algorithm that is robust to variations in data set composition (E. R. Paquet, et al., Breast Cancer Res 19, 32 (2017), the disclosure of which is incorporated herein by reference). This approach was utilized given the expected preponderance of HER2-enriched cases in this cohort.

### Correlation Analysis

The Spearman rank correlation between DSP protein data and bulk RNA data was computed for pre-treatment samples using the average of all DSP ROIs (both panCK-enriched ROIs and surrounding microenvironment-enriched ROIs) per patient. Plots showing the correlation between protein markers (FIGS. 9, 17A & 17B) were overlapped with hierarchical clustering in the form of black squares. The difference between the distribution of correlation values in pCR versus the non-pCR cases was evaluated using a two-sided Wilcoxon two-sample t-test.

### Comparative Analysis

For the comparative analyses (e.g. pre-treatment vs on-treatment, pCR vs non-pCR, panCK-enriched vs panCK-negative) of DSP protein data, where multiple regions were sampled per patient, we utilized a linear mixed-effects model with blocking by patient (D. Bates, et al., J Stat Softw 67, 1-48 (2015), the disclosure of which is incorporated herein by reference). This model allows for marker levels to be compared in a patient-matched manner while controlling for differences in the number of ROIs profiled per patient. The coefficient of the fixed effect is the change attributable to that variable (x-axis of volcano plots), and the p-value used to calculate false discovery rates (y-axis of volcano plots) is based on the t-value (a measure of the size of the difference relative to the variation in the sample data). False discovery rates (FDR) were computed using the Benjamini & Hochberg procedure (Y. Benjamini and Y. Hochberg, J R Stat Soc B 57, 289-300 (1995), the disclosure of which is incorporated herein by reference), and an FDR-adjusted p-value of 0.05 was set as the significance threshold.

### Region Subsampling

The impact of utilizing a single randomly selected region per tissue sample, rather than multiple regions, when assessing on- versus pre-treatment protein expression changes was analyzed. For these analyses (FIG. 21), 100 iterations were performed in which a single region was selected from each tissue and computed fold changes and corresponding p-values averaged over these 100 experiments. The number of random samplings was chosen empirically by raising the number of iterations beyond the number required to make the resulting output robust to further increases in the number of iterations used (p-value convergence).

### L2-Regularized Logistic Regression Using Molecular Data

Models and features: L2-logistic regression using liblinear as a solver was used for classification of pCR vs non-pCR cases. Marker values pre-treatment and on-treatment were averaged across all ROIs to derive a composite value for each marker for that timepoint. Five patients were excluded from the models because data was available only at a single timepoint (FIG. 4). Mean DSP marker expression features were used in models comparing patient timepoints, tumor versus immune markers, DSP protein features versus established predictive features (ER status and PAM50 classification), and DSP protein versus Bulk RNA features (using RNA gene transcripts corresponding to DSP protein markers). To assess heterogeneity, standard error of the mean (SEM) was calculated for marker values across all ROIs for tissues with at least 3 ROI to derive a composite value for each marker for that timepoint. These SEM features were used in combination with mean expression features in models assessing the predictive value of heterogeneity.

Model comparisons and evaluation of performance via internal cross-validation: Model performance was evaluated and models compared using nested cross-validation using the python package sklearn (F. Pedregosa, et al., J Mach Learn Res 12, 2825-2830 (2011), the disclosure of which is incorporated herein by reference). Data were divided into N folds using stratified sampling (“stratified cross-validation”). The number of folds was chosen based on the number of cases in the non-pCR group (the class with fewer cases) such that the testing data would contain two cases from each class. Each model was trained using N−1 folds and scored using mean AUROC on the remaining fold. This process was iteratively repeated holding out a different fold each time. The L2-penalization weight was chosen using stratified cross-validation within the N−1 training dataset, with the weight associated with highest mean accuracy within this inner cross-validation selected for scoring. This nested cross-validation process was repeated 100 times using randomly generated folds. Model scores were then compared using an unpaired two-sided t-test with Holm-Bonferroni correction for multiple hypotheses. ROC curves were generated by averaging across the ROC curves from the 100 repeats of N−fold cross-validation, with each repeat containing a different random split of folds.

Evaluation of model performance in an independent cohort: As described above, marker values pre-treatment and on-treatment were averaged across all ROIs to derive a composite value for each marker for that timepoint. Model selection was carried out using cross-validation as described above. The best performing model was selected and trained using the entire discovery cohort. Finally, model performance based on the AUROC was evaluated in the independent validation (test) cohort.

### Metrics of Heterogeneity

Marker heterogeneity was calculated as the mean squared error from the analysis of variance done on a linear model with marker values as the dependent variable and patient identity as the independent variable (the data set was subsetted to the particular timepoint or clinical outcome of interest).

### Perimetric Complexity

Perimetric complexity was computed for the panCK-enriched binary masks for each ROI using ImageJ (A. B. Watson, Mathematica 14 (2012), the disclosure of which is incorporated herein by reference). A linear mixed-effects model with blocking by patient was used to the compare the perimetric complexities of all the panCK-enriched regions pre-treatment and on-treatment regions and for cases that achieved a pCR versus those for cases that did not achieve a pCR.

### Multiplex IHC Analysis

Unstained, paraffin-embedded sections were analyzed by multiplex IHC analysis used the following markers: PanCK (AE1/AE2), CD8, CD45 LCA, and HER2 (29D8 CST). Stained samples were scanned, digitized as a series of square sub-images (“stamps”), and visualized using HALO. PanCK masking and tissue area masking was performed on each stamped tissue region using Fiji (ImageJ). Briefly, the PanCK channel was used to generate the masks (using the following ImageJ tools: Enhance Contrast, Threshold, Dilate, Fill Holes, Create Selection) for the panCK-positive region and the entire tissue region and CD8, CD45, and HER2 were quantified within each masked region (using the ImageJ Measure tool). A weighted average (with weights corresponding to each mask area) was used to calculate CD8, CD45, and HER2 levels across all the scanned sub-images that comprise the tissue (either tissue mask or panCK mask area).

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

1. A method of diagnostically determining pathologic complete response of a breast cancer, comprising:
obtaining measurements of expression of a set of one or more biomolecules within at least one region of interest of an on-treatment cancer biopsy, wherein the on-treatment cancer biopsy is a cancer biopsy obtained after initiation of a targeted therapy; and
determining whether targeted therapy will provide pathologic complete response in the individual utilizing a trained classifier and the biomolecule expression measurements as features in the trained classifier.

2. The method of claim 1 further comprising:
when it is determined that targeted therapy will provide pathologic complete response, administering to the individual a deescalated therapy regimen.

3. The method of claim 2, wherein the deescalated therapy regimen includes targeted therapeutic without generalized chemotherapy.

4. The method of claim 1 further comprising:
when it is determined that targeted therapy will not provide pathologic complete response, administering to the individual an escalated therapy regimen.

5. The method of claim 4, wherein the escalated therapy regimen includes a targeted therapeutic in combination with a chemotherapeutic.

6. The method of claim 4, wherein the escalated therapy regimen includes a dual-targeted therapy of two targeted therapeutics.

7. The method of claim 1, wherein the breast cancer is HER2+.

8. The method of claim 1, wherein the set of one or more biomolecules includes at least one immune response and activation biomolecule.

9. The method of claim 8, wherein the at least one immune response and activation biomolecule is CD45, CD3, CD4, CD8, CD27, CD44, CD45RO, OX40L, ICOS, Granzyme B, CD19, CD11c, CD163, CD68, CD56, CD66B, CD14, STING, PD1/PDL1, B7-H3, B7-H4, IDO-1, Lag3, or VISTA.

10. The method of claim 1, wherein the set of one or more biomolecules includes at least one cell survival biomolecule.

11. The method of claim 10, wherein the at least one cell survival biomolecule is Beta-2 microglobulin or Bcl-2.

12. The method of claim 1, wherein the breast cancer is HER2+ and wherein the set of one or more biomolecules includes at least one HER2 signaling pathway biomolecule.

13. The method of claim 12, wherein the at least one HER2 signaling pathway biomolecule is HER2, AKT/p-AKT, S6/p-S6, PTEN, p-ERK, p-STAT3.

14. The method of claim 1, wherein the set of one or more biomolecules includes an epithelial tumor tissue biomolecule.

15. The method of claim 12, wherein the at least one epithelial tumor tissue biomolecule is PanCK, Ki67, or Beta-catenin.

16. The method of claim 1, wherein the set of one or more biomolecules includes at least two of the following biomolecules: HER2, AKT/p-AKT, S6/p-S6, PTEN, p-ERK, p-STAT3, PanCK, Ki67, Beta-catenin, CD45, CD3, CD4, CD8, CD27, CD44, CD45RO, OX40L, ICOS, Granzyme B, CD19, CD11c, CD163, CD68, CD56, CD66B, CD14, STING, PD1/PDL1, B7-H3, B7-H4, IDO-1, Lag3, VISTA, Beta-2 microglobulin or Bcl-2.

17. The method of claim 1, wherein the set of one or more biomolecules includes CD45.

18. The method of claim 1, wherein the cancer is HER2+ and wherein the set of one or more biomolecules includes HER2.

19. The method of claim 1, wherein the cancer biopsy is formalin fixed paraffin embedded, OCT embedded, or flash frozen.

20. The method of claim 1, wherein the at least one region of interest is determined by pancytokeratin-positive (panCK+) tumor cells or CD45-positive (CD45+) immune cells.

21.-39. (canceled)
